(19) United States
(12) Patent Application Publication
(10) Pub. No.: US 2003/0109468 A1
(43) Pub. Date: Jun. 12, 2003

(54) Novel compounds that enhance dimerization of the subunits of HIV-1 reverse transcriptase having mutations associated with resistance to nonnucleoside reverse transcriptase inhibitors

(76) Inventor: Bryan M. O' Hara, Norwood, NJ (US)

Correspondence Address: Kathryn M. Brown, PROGENICS PHARMACEUTICALS, INC., 777 Old Saw Mill River Road, Tarrytown, NY, 10591, US

(21) Appl. No.: 10/010,129

(22) Filed: Dec. 6, 2001

(51) Int. Cl. A61K 31/00; C12Q 1/70; A01N 61/00; A61K 31/70; A01N 43/04; C12P 19/34; A61K 39/12; A61K 39/21; C12N 7/00; C12N 7/01; C12N 15/00; C12N 15/09; C12N 15/63; C12N 15/70; C12N 15/74; C12N 5/00; C12N 5/02; C07K 16/00; C12P 21/08

(52) U.S. Cl. 514/44; 514/1; 435/5; 435/91.1; 435/91.33; 435/235.1; 435/325; 424/208.1; 424/199.1; 435/320.1; 530/388.75; 530/388.8

(57) ABSTRACT

The invention provides novel compounds that enhance dimerization of the subunits of HIV-1 reverse transcriptase having mutations associated with resistance to nonnucleoside reverse transcriptase inhibitors (NNRTIs). Such compounds inhibit HIV-1 reverse transcriptase activity and as such, inhibit HIV-1, in particular HIV-1 resistant to conventional NNRTIs. The invention provides a method of determining whether a compound enhances formation of a complex between a p66 and p51 subunit polypeptides of HIV-1 reverse transcriptase, in which the p66 subunit has at least one or more mutations associated with resistance of HIV-1 to at least one NNRTI. The invention further provides methods of using a compound that enhances formation of a complex between a p66 subunit polypeptide having at least one mutation associated with resistance of HIV-1 to at least one NNRTI and a p51 subunit of HIV-1 reverse transcriptase.

### FIELD OF THE INVENTION

The invention relates generally to compounds that inhibit Human Immunodeficiency Virus (HIV-1). More specifically, the invention relates to compounds that inhibit wild-type HIV-1 and HIV-1 resistant to at least one nonnucleoside reverse transcriptase inhibitor. In particular, the invention relates to compounds that enhance dimerization of the p66 subunit having at least one mutation associated with resistance and the p51 subunit of HIV-1 reverse transcriptase. The invention further relates to methods of screening for compounds, as well as uses of the compounds.

### BACKGROUND OF THE INVENTION

Nonnucleoside reverse transcriptase inhibitors (NNRTIs) are allosteric inhibitors of the human immunodeficiency virus type 1 (HIV-1) reverse transcriptase (RT). Yeast grown in the presence of many of these drugs exhibited dramatically increased association of the p66 and p51 subunits of the HIV-1 RT as reported by a yeast two-hybrid assay. The enhancement required drug binding by RT; introduction of a drug-resistance mutation into the p66 construct negated or reduced the enhancement effect. The drugs could also induce heterodimerization of dimerization defective mutants. Coimmunoprecipitation of RT subunits from yeast lysates confirmed the induction of heterodimer formation by the drugs. In vitro binding studies indicate that NNRTIs can bind tightly to p66 but not p51, and then mediate subsequent heterodimerization.

The human immunodeficiency virus type 1 (HIV-1) reverse transcriptase (RT) catalyzes the conversion of genomic RNA into double-stranded proviral DNA after cell entry (2^{nd} series-1), utilizing the RNA- and DNA-dependent polymerase and ribonuclease H (RNase H) activities of the enzyme. The HIV-1 RT is an asymmetric dimer consisting of p66 and p51 polypeptides (2^{nd} series-2, 3). The p51 subunit contains the identical N-terminal sequences as p66, but lacks the C-terminal RNase H domain. The structure of the HIV-1 RT has been elucidated by X-ray crystallography in several forms including the unliganded enzyme (2^{nd} series-4), in complex with nonnucleoside reverse transcriptase inhibitors (NNRTIs) (2^{nd} series-5, 6) and bound to template-primer with (2^{nd} series-7) or without dNTP substrate (2^{nd} series-8). The polymerase domain of the p66 subunit resembles a right hand and contains the fingers, palm, thumb and connection subdomains, with the latter acting as a tether between the polymerase and RNase H regions (2^{nd} series-5, 8). Although p51 has the same polymerase domains as p66, the relative orientations of these individual domains differ markedly (2^{nd} series-5, 8). Structural analysis reveals three major contacts between p66 and p51, with most of the interaction surfaces being hydrophobic (2^{nd} series-9, 10).

NNRTIs are chemically diverse, largely hydrophobic compounds which comprise over 30 different classes (2^{nd} series-11, 12). NNRTIs do not require intracellular metabolism for activity, are noncompetitive inhibitors of RT activity with respect to dNTP substrate and template/primer, and are relatively noncytotoxic (2^{nd} series-11). NNRTIs bind to a hydrophobic pocket close to but distinct from the polymerase active site in the p66 subunit (2^{nd} series-13, 14) and inhibit enzyme activity by mediating allosteric changes in the RT (2^{nd} series-15, 16). Initial clinical use of NNRTIs as monotherapy and selection of drug-resistant variants in cell culture results in the rapid emergence of highly drug-resistant variants due to single amino acid changes (2^{nd} series-17, 18) in the NNRTI binding pocket that directly affect drug binding (2^{nd} series-13, 14). The NNRTIs currently approved for use in highly active antiretroviral therapy include nevirapine (2nd series-19), delavirdine (2^{nd} series-20) and efavirenz (2^{nd} series-21).

Efavirenz (also known as DMP 266 or SUSTIVA™) is a potent nonnucleoside inhibitor of HIV-1 reverse transcriptase (RT) activity and of HIV-1 replication in vitro and in vivo. Most patients on efavirenz-containing regimes have sustained antiviral responses; however, rebounds in plasma viral load have been observed in some patients in association with the emergence of mutant strains of HIV-1. Bacheler, L. et al have reported genotypic mutations in the RT gene that appear to correlate with phenotypic resistance to efavirenz in virus isolates from patients failing NNRTI therapy (J. Virology, Vol 75, No. 11, pp4999-5008, June 2001). Thus, there is a need in the art for novel compounds effective against emerging mutations in the RT gene, in particular for use in patients failing conventional NNRTI therapy.

It has been reported that HIV-1 RT heterodimerization can be effectively monitored in the yeast two-hybrid (Y2H) system using appropriately engineered constructs (2^{nd} series-22). This system has been used to assess the effect of NNRTIs on the β-galactosidase β-gal) readout in yeast. Several NNRTIs induced dramatic increases in β-gal activity and this increase was due to enhanced association between the RT subunits as a result of a specific interaction of drug with the p66 subunit (Tachedjian, G. et al Proc. Natl. Acad. Sci Vol 98, No 13, pp7188-7193, Jun. 19, 2001). The yeast two-hybrid system has been modified in the present invention and used to identify novel NNRTIs or mimics of NNRTIs that enhance HIV-1 RT dimerization, in particular, compounds that enhance both wild-type HIV-1 RT and enhance HIV-1 RT having mutations associated with resistance of HIV-1 to known NNRTIs, such as efavirenz, nevirapine, and delavirdine.

### SUMMARY OF THE INVENTION

The present invention provides a method of determining whether a compound enhances formation of a complex between a wild-type p66 subunit polypeptide of HIV-1 reverse transcriptase and a wild-type p51 subunit polypeptide of HIV-1 reverse transcriptase and/or enhances formation of a complex between a p66 subunit having at least one mutation associated with resistance of HIV-1 to at least one nonnucleoside reverse transcriptase inhibitor (NNRTI) and a wild-type p51 subunit of HIV-1 reverse transcriptase, said method comprises:

a) contacting a yeast cell with the compound, which cell comprises (i) a first plasmid which expresses a fusion protein comprising a wild-type p66 subunit polypeptide of HIV-1 reverse transcriptase, (ii) a second plasmid which expresses a fusion protein comprising a wild-type p51 subunit polypeptide of HIV-1 reverse transcriptase, and (iii) a reporter gene which is activated in the presence of a complex between the wild-type p66 subunit polypeptide and the wild-type p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the wild-type p51 subunit polypeptide of HIV-1 reverse transcriptase and the wild-type p66 subunit polypeptide of HIV-1 reverse transcriptase; and

c) contacting a yeast cell with the compound, which cell comprises (i) a third plasmid which expresses a fusion protein comprising a p66 subunit polypeptide of HIV-1 reverse transcriptase having at least one mutation associated with resistance of HIV-1 to at least one NNRTI, (ii) a second plasmid which expresses a fusion protein comprising a wild-type p51 subunit polypeptide of HIV-1 reverse transcriptase, and (iii) a reporter gene which is activated in the presence of a complex between the p66 subunit polypeptide having at least one mutation and the wild-type p51 subunit polypeptide, and determining the level of activity of the reporter gene in the cell in the presence of the compound; and

d) comparing the level of activity of the reporter gene determined in step (c) with a level of activity of the reporter gene determined in the absence of the compound, wherein an increased level of activity of the reporter gene determined in step (c) indicates that the compound is an activator of the formation of the complex between the wild-type p51 subunit polypeptide of HIV-1 reverse transcriptase and the p66 subunit polypeptide of HIV-1 reverse transcriptase having at least one mutation associated with resistance of HIV-1 to at least one NNRTI.

The present invention further provides a method of determining whether a compound is capable of inhibiting wild-type HIV-1 reverse transcriptase activity and/or mutant HIV-1 reverse transcriptase activity, said method comprises:

a) contacting a yeast cell with the compound, which cell comprises (i) a first plasmid which expresses a fusion protein comprising a wild-type p66 subunit polypeptide of HIV-1 reverse transcriptase, (ii) a second plasmid which expresses a fusion protein comprising a wild-type p51 subunit polypeptide of HIV-1 reverse transcriptase, and (iii) a reporter gene which is activated in the presence of a complex between the wild-type p66 subunit polypeptide and the wild-type p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the wild-type p51 subunit polypeptide of HIV-1 reverse transcriptase and the wild-type p66 subunit polypeptide of HIV-1 reverse transcriptase, thereby indicating that the compound is capable of inhibiting wild-type HIV-1 reverse transcriptase activity; and

c) contacting a yeast cell with the compound, which cell comprises (i) a third plasmid which expresses a fusion protein comprising a p66 subunit polypeptide of HIV-1 reverse transcriptase having at least one mutation associated with resistance of HIV-1 to at least one NNRTI, (ii) a second plasmid which expresses a fusion protein comprising a wild-type p51 subunit polypeptide of HIV-1 reverse transcriptase, and (iii) a reporter gene which is activated in the presence of a complex between the p66 subunit polypeptide having at least one mutation and the wild-type p51 subunit polypeptide, and determining the level of activity of the reporter gene in the cell in the presence of the compound; and

d) comparing the level of activity of the reporter gene determined in step (c) with a level of activity of the reporter gene determined in the absence of the compound, wherein an increased level of activity of the reporter gene determined in step (c) indicates that the compound is an activator of the formation of the complex between the wild-type p51 subunit polypeptide of HIV-1 reverse transcriptase and the p66 subunit polypeptide of HIV-1 reverse transcriptase having at least one mutation associated with resistance of HIV-1 to at least one NNRTI, thereby indicating that the compound is capable of inhibiting mutant HIV-1 reverse transcriptase activity.

The method of the present invention further provides a p66 subunit polypeptide of reverse transcriptase comprising one or more mutations, said mutations associated with resistance of HIV-1 to at least one NNRTI, said mutations selected from the group consisting of F227L, G190A, G190E, G190S, K101E, K103N, K238T, L100I, P225H, V106A, V106I, V108I, Y181C, Y188H, Y188L, and combinations thereof.

An object of the invention is a compound determined to be capable of enhancing formation of a complex between a p66 subunit polypeptide of HIV-1 reverse transcriptase having at least one mutation and a wild-type p51 subunit polypeptide of HIV-1 reverse transcriptase.

Another object of the invention is a compound determined to be capable of enhancing formation of a complex between a p66 subunit polypeptide of HIV-1 reverse transcriptase having multiple mutations and a wild-type p51 subunit polypeptide of HIV-1 reverse transcriptase, each mutation is associated with an increase in resistance of HIV-1 to at least one NNRTI.

Yet another object of the invention is a compound determined to be capable of enhancing formation of a complex between a p66 subunit polypeptide of HIV-1 reverse transcriptase and a p51 subunit polypeptide of HIV-1 reverse transcriptase, wherein the reverse transcriptase has at least one mutation selected from the group consisting of F227L, G190A, G190E, G190S, K101E, K103N, K238T, L100I, P225H, V106A, V106I, V108I, Y181C, Y188H, Y188L, and combinations thereof.

Another object of the invention is a compound which provides enhancement of formation of complex between a p66 subunit having at least one mutation associated with an increase in resistance of HIV-1 to at least one NNRTI and a wild-type p55 subunit which is greater than the enhancement of formation of complex between a wild-type p66 subunit and a wild-type p55 subunit.

A further object of the invention is a compound which provides enhancement of formation of a complex between a p66 subunit having at least one mutation and a wild-type p55 subunit which is higher than the enhancement of complex formation in the presence of a known NNRTI at a given concentration, that concentration being in a linear range of enhancement for both the compound and the known NNRTI.

Another object of the invention is a compound capable of inhibiting HIV-1 in which the inhibition is characterized by enhancing formation of a complex between a p66 subunit polypeptide and a p51 subunit polypeptide of reverse transcriptase of the HIV-1, in which the HIV-1 is a wild-type or the HIV-1 has at least one mutation associated with resistance to at least one NNRTI.

This invention provides a method of making a pharmaceutical composition which comprises:

a) determining whether a compound enhances formation of a complex by one of the methods described herein;

b) recovering the compound; and

c) admixing the compound with a pharmaceutically acceptable carrier.

This invention provides a method of enhancing formation of a complex between the p66 subunit polypeptide of HIV-1 reverse transcriptase and a p51 subunit polypeptide of HIV-1 reverse transcriptase, which comprises contacting either (1) the p66 subunit polypeptide, or (2) both the p66 subunit polypeptide and the p51 subunit polypeptide, with an effective amount of a compound of the present invention, so to thereby enhance formation of a complex between the p66 subunit polypeptide of HIV-1 reverse transcriptase and a p51 subunit polypeptide of HIV-1 reverse transcriptase, wherein the reverse transcriptase is wild-type or has at least one mutation associated with resistance to at least one NNRTI.

Another aspect of the invention is a method of enhancing formation of a complex between a p66 subunit polypeptide of HIV-1 reverse transcriptase and a p51 subunit polypeptide of reverse transcriptase, in which the HIV-1 reverse transcriptase is present in a subject and the contacting is effected by administering the compound to the subject in an amount effective to enhance formation of a complex.

Yet another aspect of the invention is a method of treating infection by HIV-1 or for treating AIDS or ARC in a subject comprising administration of an amount of at least one compound of the present invention capable of inhibiting HIV-1, wherein the HIV-1 is wild-type or the HIV-1 has at least one mutation associated with resistance of at least one NNRTI and said amount is effective to treat the infection or to treat AIDS.

### BRIEF DESCRIPTION OF THE FIGURES

FIG. 1

RT fusion constructs, encoded fusion proteins and expression of fusions in yeast reporter strains. The six-alanine linker is denoted by the hatched box, and the HA epitope by black shaded regions. p66 and p51 indicate the 66 kDa and 51 kDa subunits of HIV-1 RT, respectively. Expression of fusion proteins was determined by introducing the indicated plasmids into CTY10-5d, except for p66 GBT9 and p51AS2-1 which were introduced into HF7c. Fusion protein expression was detected by probing yeast protein lysates with anti-RT antibodies as described in the Materials and Methods. ++, high; +, moderate; +/−, low and −, undetectable protein expression. ND, not done.

FIG. 2

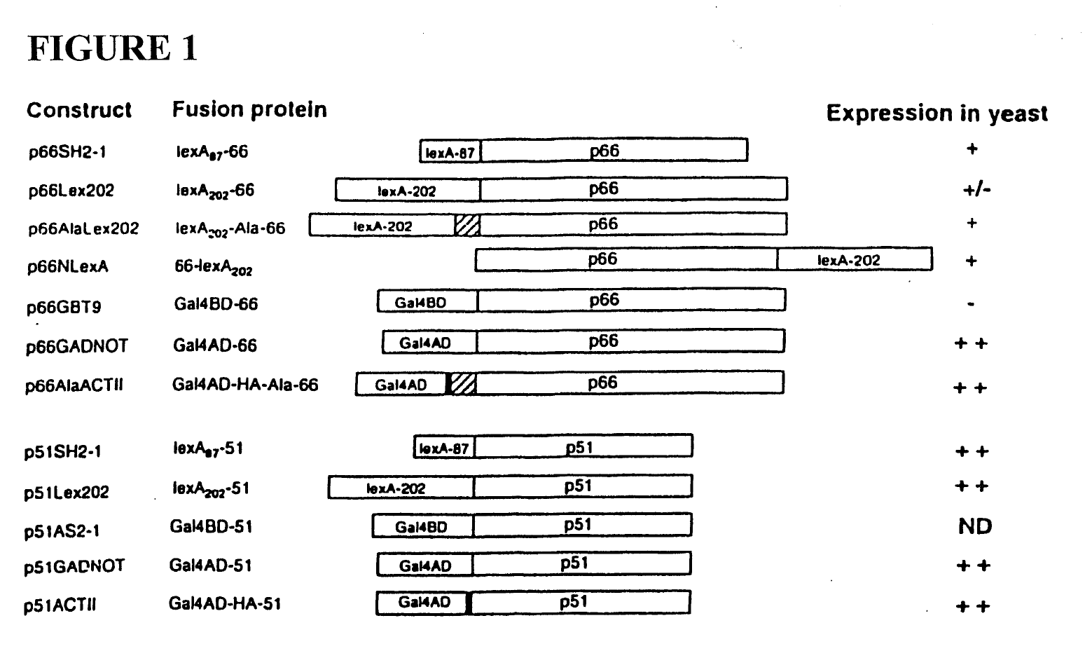

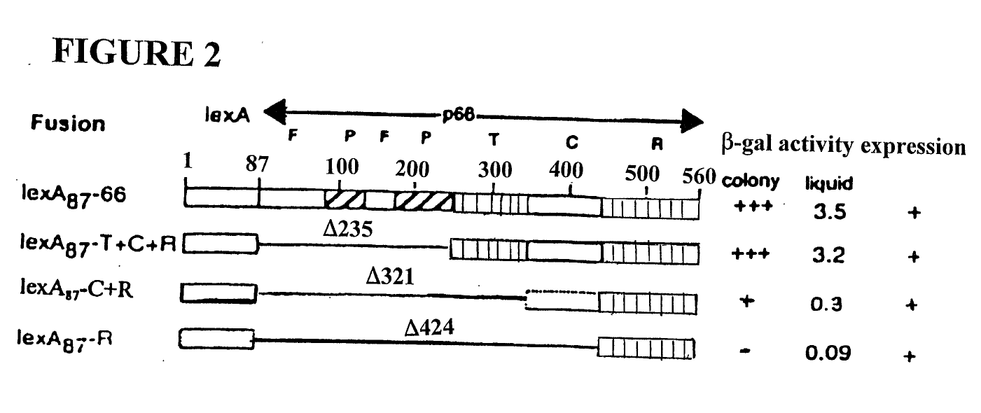

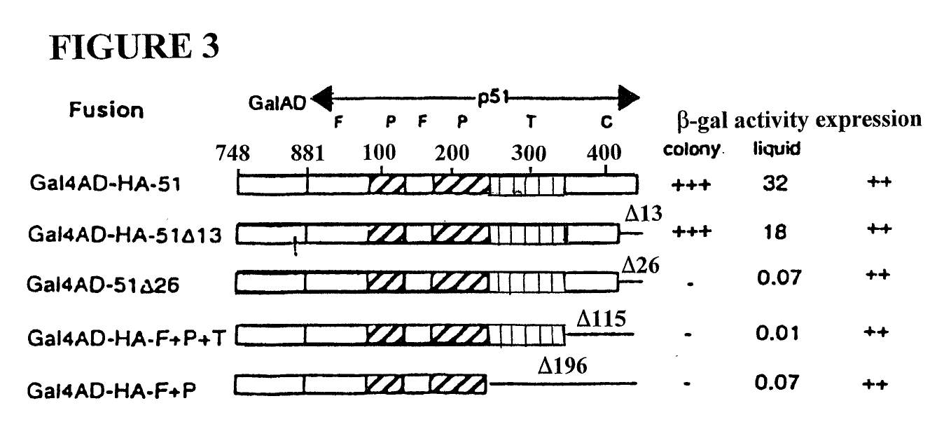

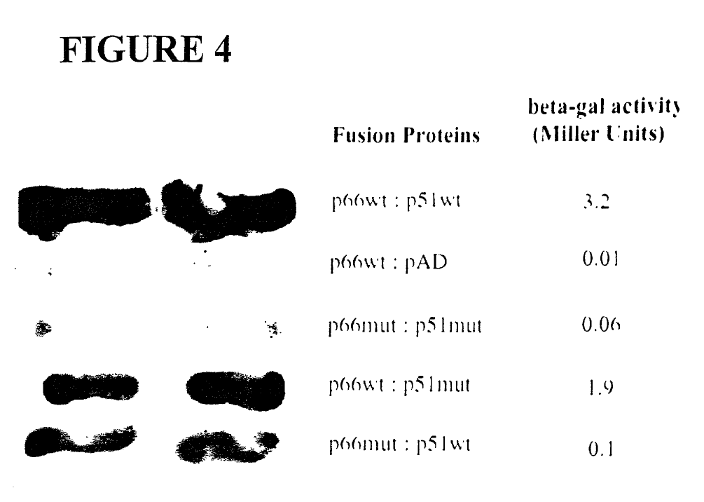

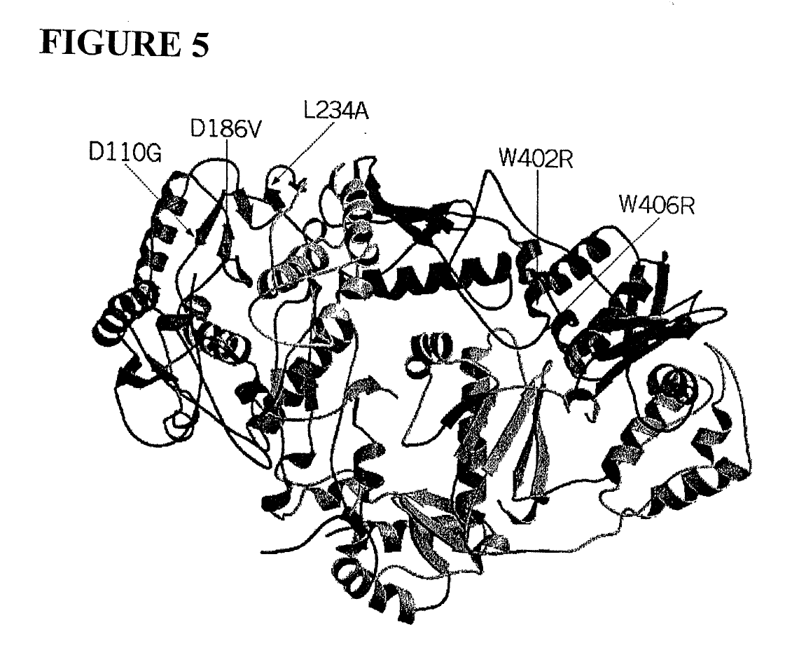

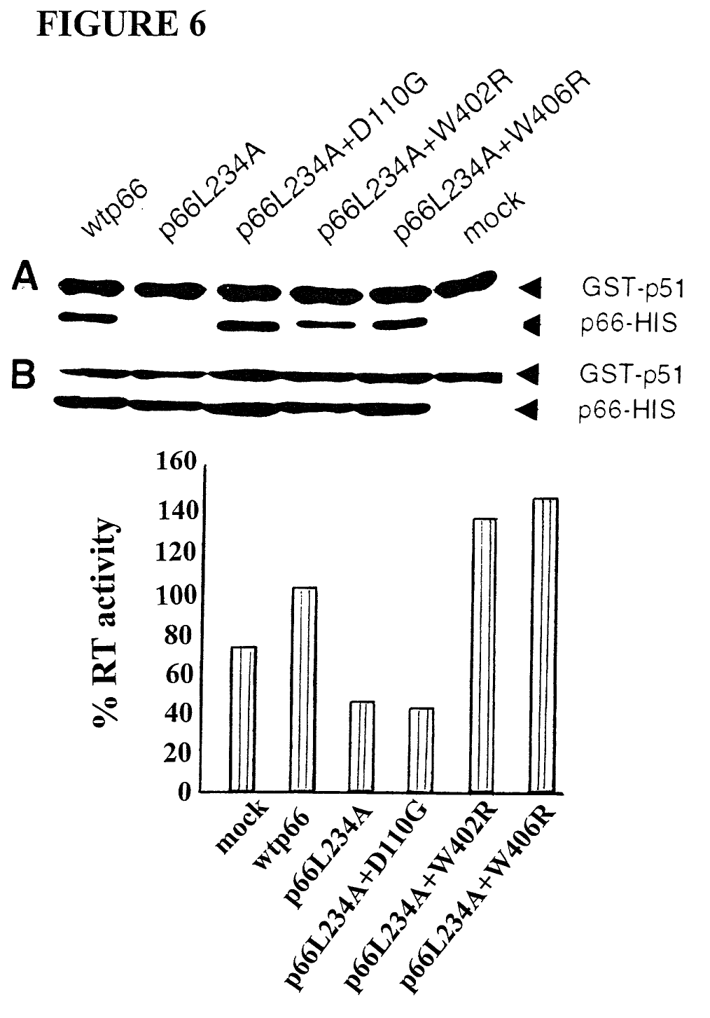

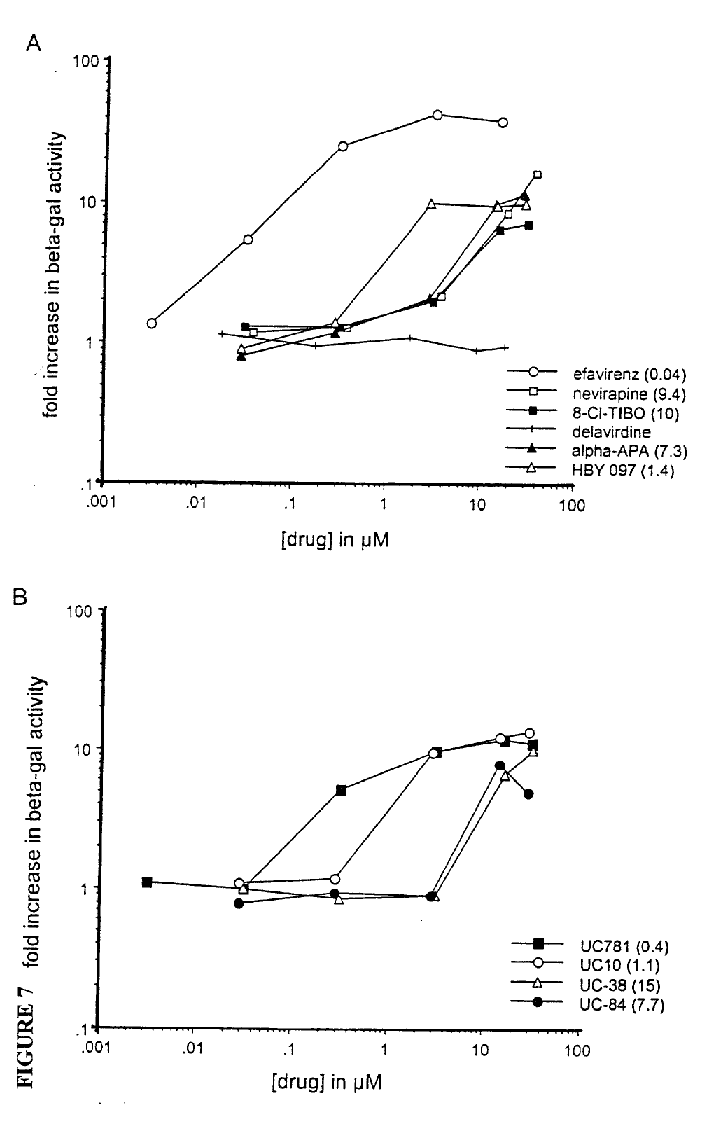

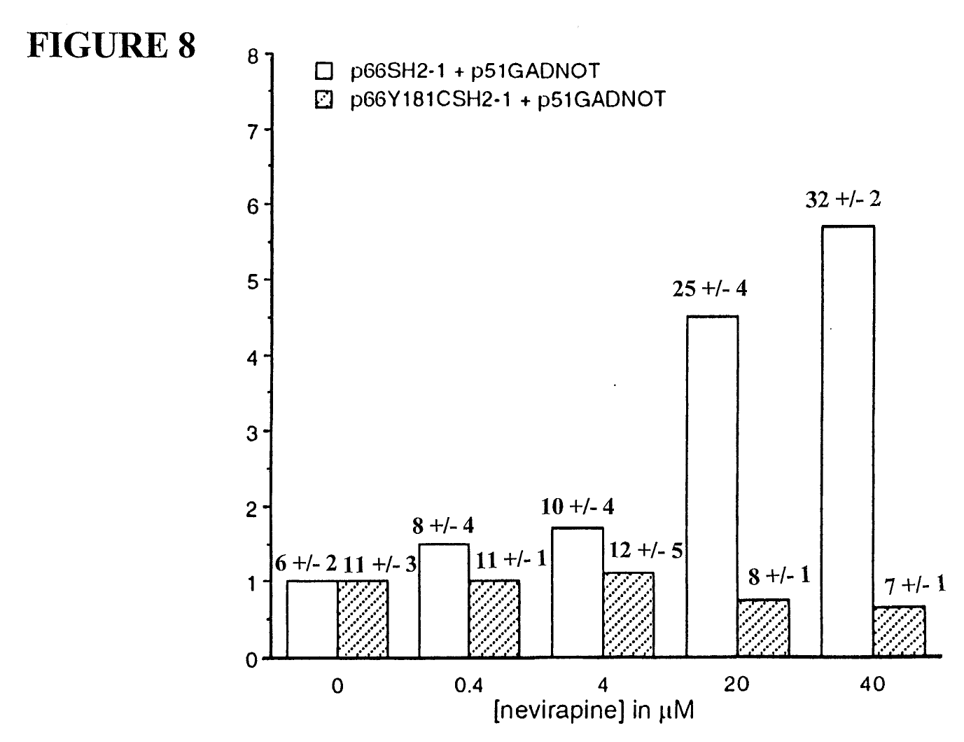

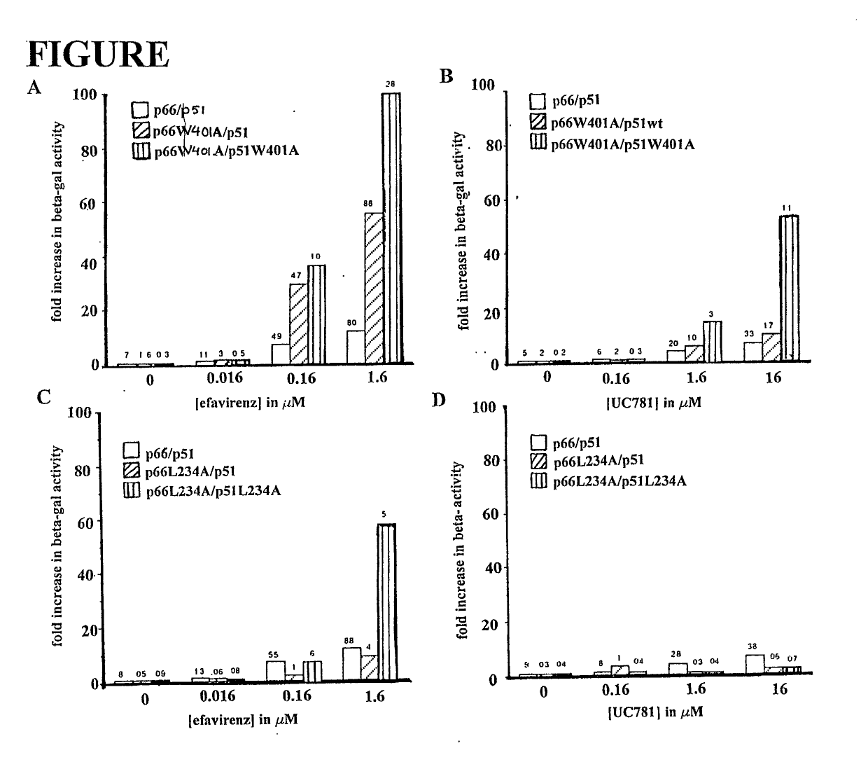

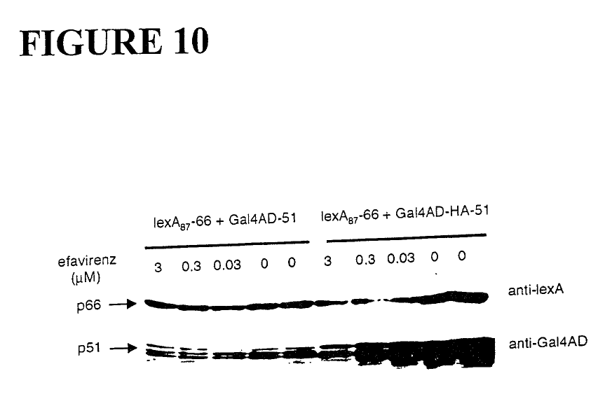

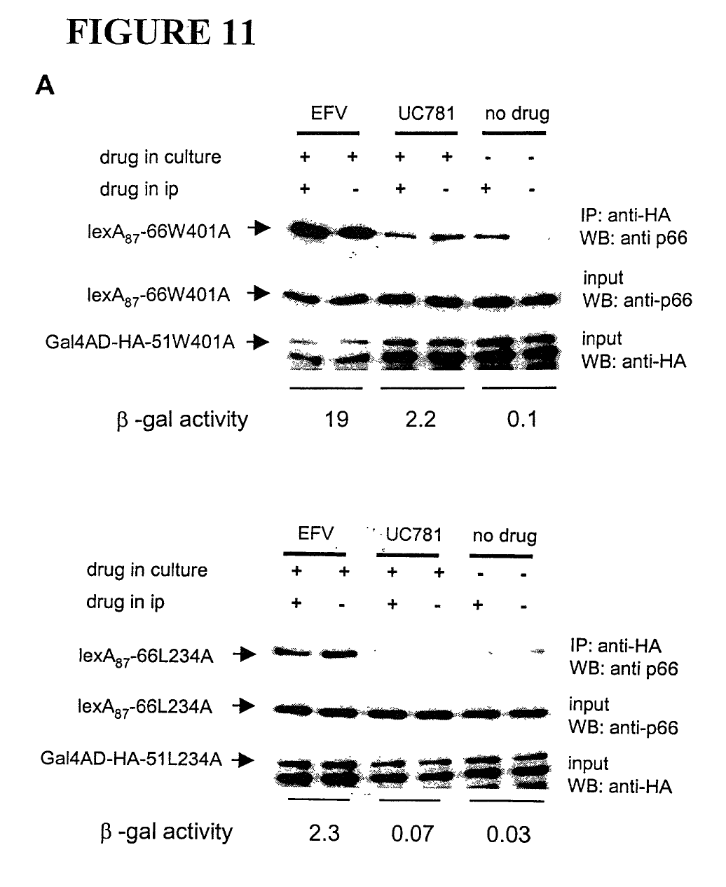

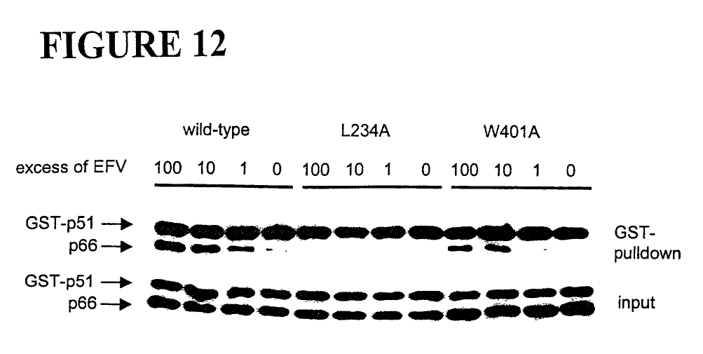

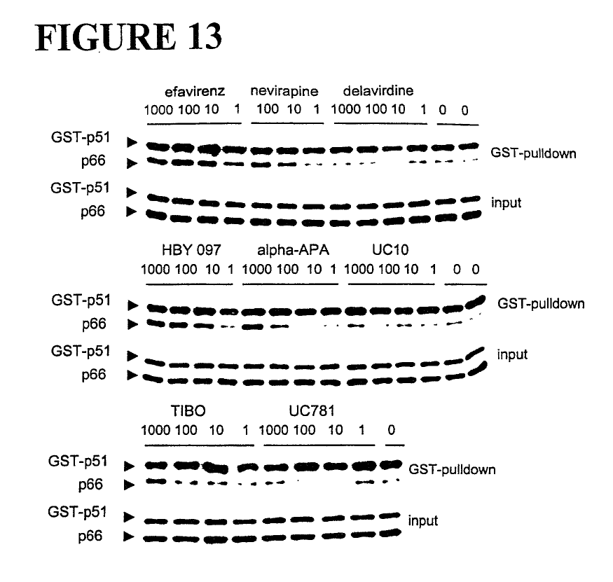

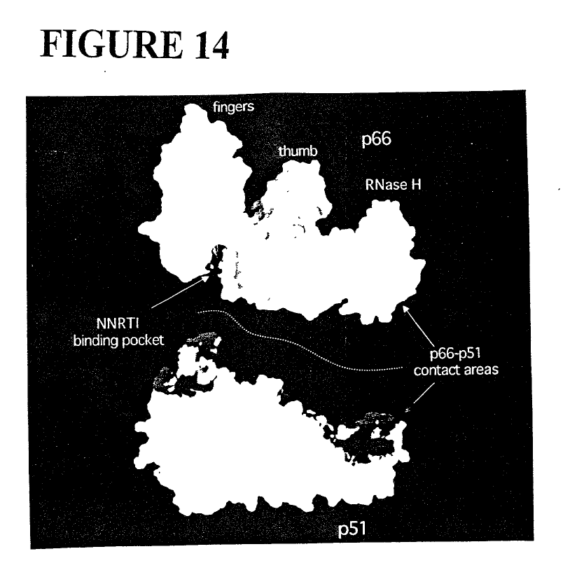

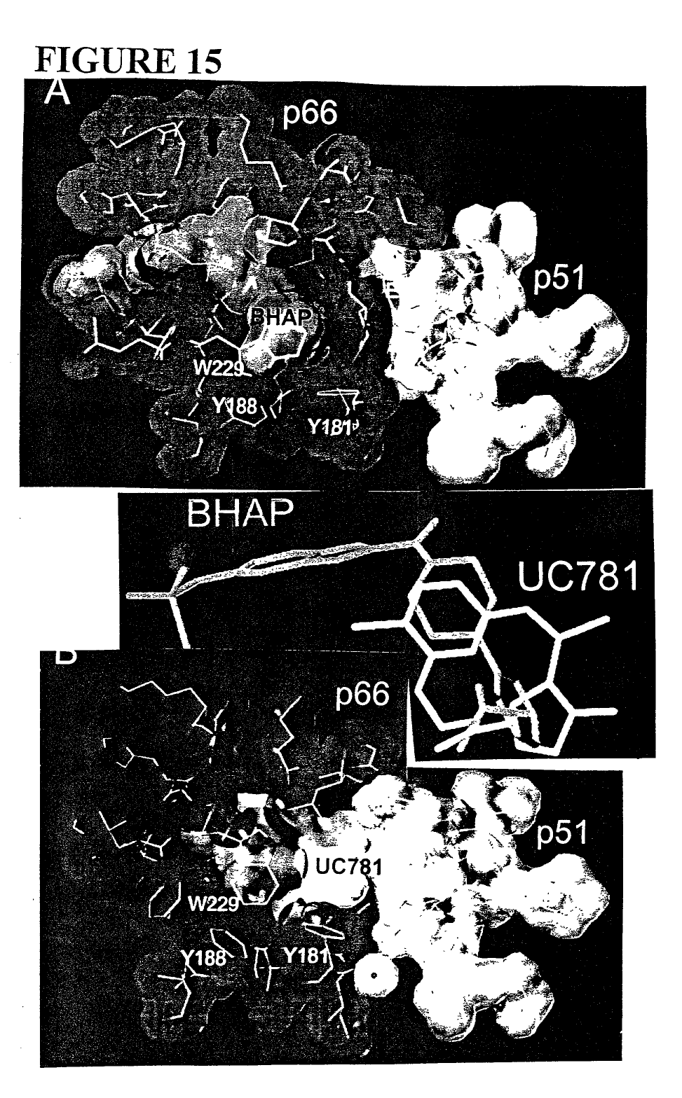

Interaction of p66 deletion mutants with Gal4AD-HA-51 fusion protein. p66 polymerase domains were fused to the C-terminus of lexA87 in pSH2-1. CTY10-5d was cotransformed with the appropriate constructs. Transformants were lifted onto nitrocellulose and subjected to β-gal colony lift assay to determine intensities of blue color produced as defined in Tables 1 and 2. β-gal activity from liquid assays is expressed in Miller Units. Expression in CTY10-5d of p66 fusion proteins was detected using anti-lexA polyclonal antibodies. Expression levels are as defined as in the legend for FIG. 1.

FIG. 3

Interaction of C-terminal deletion mutants of p51 with lexA202-Ala-66. p51 domains were fused to the C-terminus of the Gal4AD in PACTII. Deletions at the C-terminus are denoted by the number of amino acids missing from the end of p51. β-gal activity was determined as described in the legend of FIG. 2. Expression of p51 fusion proteins in CTY10-5d was detected using anti-GAL4AD antibodies, and expression levels are as denoted in the legend for FIG. 1.

FIG. 4

L234A inhibits RT dimerization in the Y2H assay. CTY10-5d was cotransformed with expression constructs, and yeast patches were subjected to both the β-gal colony lift and liquid assays. The green is hydrolyzed X-gal and reflects β-gal activity. p66 wt and p51wt denote wild-type lex202-Ala-HX66 and Gal4AD-HX51 fusion proteins, respectively. pAD denotes PGADNOT. p66mut and p51mut denote RT fusion proteins lex202-Ala-66-L234A and Gal4AD-51-L234A, respectively.

FIG. 5

Ribbon diagram of unliganded HIV-1 RT showing position of L234A primer grip mutation and locations of suppressors (shaded black). The figure was generated by MOLSCRIPT (38) and RASTER3D (39) with coordinates (2) retrieved from the Research Collaboratory for Structural Bioinformatics (RCSB) Protein Data Bank (PDB) (http://www.rcsb.org/pdb, PDB ID: 1HMV.pdb). Domains are defined as in (3) with fingers, blue; palm, green; thumb, yellow; connection, red; and RNase H in purple. Domains in p66 are in fully saturated colors, whereas in p51 they have decreased saturation. Secondary structure was assigned using DSSP (40). Spirals represent alpha-helices, arrows denote beta-strands.

FIG. 6

In vitro assay for binding of GST-p51 and p66 to form active RT heterodimers. Panel A: Bacterial lysates containing GST-p51 and various p66 proteins as indicated were incubated overnight and captured on glutathione beads. The complexes were eluted, resolved by SDS-PAGE, blotted to membrane and detected by monoclonal anti-RT antibodies. Mock, GST-p51 alone. Panel B: An aliquot of each incubation mix, reflecting input protein, was directly analyzed by SDS-PAGE and Western blot as in Panel A. Panel C: Bound proteins were eluted with glutathione and assayed for RT activity with homopolymeric template-primer. Values are normalized to the wild-type control.

FIG. 7

Dose-response curve showing the enhancement by NNRTIs of B-gal activity in yeast cotransformed with lexA87-66 and Gal4AD-51. The fold increase in β-gal activity was calculated by dividing β-gal activity (in Miller Units) for each drug concentration with the β-gal activity from cells grown in the absence of inhibitor. The data represents the average results from two independent experiments. The concentration of drug that mediates a 5-fold increase in B-gal activity is shown in parenthesis. A: β-gal enhancement activity of the NNRTIs, efavirenz, HBY 097, β-APA, nevirapine, 8-Cl-TIBO and delavirdine. B: B-gal enhancement activity of the carboxanilide class of NNRTIs.

FIG. 8

Effect of the Y181C mutation on enhancement of β-gal activity in yeast by nevirapine. Yeast expressing wild-type lexA87-66 and Gal4AD-51 or mutant lexA87-66Y181C and wild type Gal4AD-51 were grown in the presence of nevirapine and assayed for B-gal activity. Results are expressed as fold increase in B-gal activity compared to untreated cells. Values on top of each bar indicates β-gal activity (in Miller Units)+/−standard deviation.

FIG. 9

Effect of efavirenz on β-gal activity in yeast expressing the dimerization defective mutants L234A and W401A. Yeast expressing wild-type p66 bait and p51 prey fusions, mutant p66 bait and wild-type p51 prey and mutant p66 bait and mutant p51 prey fusions were assayed for β-gal activity. Results are expressed as the fold increase in β-gal activity compared to untreated controls. Values on top of each bar indicates β-gal activity in Miller Units. Effect of efavirenz on yeast expressing bait and prey fusions with the W401A change (A) or L234A change (B).

FIG. 10

Coimmunoprecipitaton assay detecting heterodimer formation in yeast propagated in the presence of NNRTIs. (A): Yeast expressing p66 bait and p51 prey fusions containing the W401A mutation were grown in the presence of efavirenz (EFV), UC781 or no drug. After growth, yeast were processed in the absence or presence of added drug (drug in ip). Heterodimers present in lysates were detected by immunoprecipation of Gal4AD-HA-51W401A with anti-HA antibodies followed by immunodetection of coimmunoprecipitated p66. The β-gal activity for each treatment was determined and expressed in Miller Units. (B). Yeast expressing p66 bait and p51 prey fusions containing the L234A mutation.

FIG. 11

Western blot analysis of HIV-1 RT heterodimers formed in the presence of efavirenz (EFV) in vitro. Bacteria expressing either wild-type p66-His and GST-p51, or dimerization defective mutants were induced and lysates were prepared. Lysates were mixed and incubated overnight at 4° C. with or without drug and dimers were captured by binding to Glutathione Sepharose 4B beads. Heterodimer bound to beads were resolved by SDS-PAGE and proteins detected by probing with anti-RT monoclonal antibodies.

FIG. 12

Western blot analysis of HIV-1 RT heterodimers formed in the presence of NNRTIs in vitro. Bacterially expressed proteins p66-His and GST-p51 were combined in the presence of 1 1000 fold molar excess of drug and incubated overnight. Heterodimers were captured and detected as described in the legend of FIG. 11.

FIG. 13

Western blot analysis of heterodimer formation after pretreatment of one of the subunits with efavirenz. p66His, GST-p51 and M15 bacterial lysate were preincubated in the absence or presence of 10-1000 fold molar excess of efavirenz. Lysates were washed and the presence of residual efavirenz was assayed by the addition of GST-p51, p66-His or both subunits, respectively. Heterodimers were captured and detected as described in the legend of FIG. 11.

FIG. 14

Molecular surface representation of the p66 and p51 subunits of HIV-1 RT. Residues colored yellow (p66) or magenta (p51) are amino acids that are not accessible to solvent in the presence of the other subunit in the heterodimeric form. The NNRTI binding pocket is shown in red. The sum of the surface areas colored in yellow and magenta is the total buried surface area at the interface of the two subunits.

FIG. 15

Binding of delavirdine (BHAP) (A) and UC781 (B) at the interface of the p66 (magenta) and p51 (yellow) subunits of the HIV-1 RT. Delavirdine, a large inhibitor, is bound further away from the p66/p51 interface. The relative orientation of the inhibitors in the NNRTI binding pocket is shown in (C). Some residues that comprise the NNRTI binding site have been omitted for clarity.

### DETAILED DESCRIPTION OF THE INVENTION

The present invention relates to compounds that inhibit HIV-1. The invention further provides compounds that inhibit HIV-1 resistant to at least one nonnucleoside reverse transcriptase inhibitor (NNRTI), as well as inhibit wild-type HIV-1. The compound of the present invention inhibit HIV-1 having one or more mutations in the reverse transcriptase gene, the mutations associated with resistance to one or more NNRTIs. The novel compounds of the present invention are useful in the inhibition of HIV-1, in particular through the inhibition of HIV-1 reverse transcriptase by enhancing dimerization of the p66 subunit polypeptide of reverse transcriptase with the p51 subunit polypeptide of reverse transcriptase or by otherwise conformationally altering the protein thus preventing or inhibiting RT activity. The compound of the present invention may act at the nonnucleoside reverse transcriptase binding pocket or may act at a location on HIV-1 reverse transcriptase distinct from the nonnucleoside reverse transcriptase binding pocket.

The present invention provides novel compounds determined by the methods described herein to be capable of enhancing formation, or enhancing dimerization of a complex between a p66 subunit polypeptide of HIV-1 reverse transcriptase having at least one mutation associated with resistance to at least one NNRTI and a wild-type p51 subunit polypeptide of HIV-1 reverse transcriptase. The novel compounds of the present invention inhibit wild-type HIV-1 and have the added advantage of inhibiting HIV-1 having drug resistance to one or more nonnucleoside reverse transcriptase inhibitors (NNRTIs). The NNRTI drug-resistant HIV-1 has one or more mutations in the reverse transcriptase gene resulting in expression of a mutant p66 subunit polypeptide having resistance to at least one NNRTI, as well as expression of a mutant p51 subunit polypeptide having the corresponding mutation.

The novel compounds enhance dimerization of a complex between a p66 subunit polypeptide of HIV-1 reverse transcriptase having one or more mutations, each mutation being associated with an increased resistance of HIV-1 to at least one NNRTI. The p66 subunit may have two mutations, three mutations, four mutations, five or more mutations associated with increased resistance of HIV-1 to at least one NNRTI.

In one embodiment of the present invention, the compound enhances formation of a complex between a p66 subunit having one or more mutations selected from the group of mutations consisting of F227L, G190A, G190E, G190S, K101E, K103N, K238T, L1001, P225H, V106A, V106I, V108I, Y181C, Y188H, Y188L, and combinations thereof, and a p51 subunit polypeptide of HIV-1 reverse transcriptase.

In one embodiment, the mutation is a single mutation, in particular, a mutation in K103N or Y188L.

In one particular embodiment of the present invention, the compound enhances formation of a complex between a p66 subunit having two mutations, the mutations selected from the group consisting of L1001 and K103N; Y181C and K103N; K101E and K103N; K103N and Y188H; V106I and Y188L; K103N and P225H; K103N and V108I, and the like, and p51 subunit of HIV-1 reverse transcriptase.

In another embodiment, the mutation is a double mutation, wherein a primary mutation is K103N or Y188L and a secondary mutation is selected from the group consisting of V106I, V108I, Y181C, Y188H, P225H, and F227L.

In one embodiment of the present invention, the compound enhances formation of a complex between a p66 subunit polypeptide of HIV-1 reverse transcriptase having at least one mutation associated with an increased resistance of HIV-1 to at least one NNRTI and a wild-type p55 subunit polypeptide of HIV-1 reverse transcriptase comparably to an enhancement of formation of a complex between a wild-type p66 subunit polypeptide of HIV-1 reverse transcriptase and a wild-type p55 subunit polypeptide of reverse transcriptase at a given compound concentration. For such a compound, the enhancement obtained using the p66 subunit having at least one mutation associated with an increase in resistance of HIV-1 to at least one NNRTI and the enhancement obtained using wild-type p66 are within plus-or-minus 15%.

In another embodiment of the present invention, the compound enhances formation of a complex between a p66 subunit having at least one mutation associated with resistance of HIV-1 to at least one NNRTI and a wild-type p51 subunit of HIV-1 reverse transcriptase and the compound has little or no enhancement of formation of complex between the wild-type p66 subunit polypeptide and the wild-type p55 subunit polypeptide.

In another embodiment, the enhancement of formation of complex between the p66 subunit having at least one mutation associated with an increase in resistance of HIV-1 to at least one NNRTI and a wild-type p55 subunit by the compound of the present invention is greater than the enhancement of formation of complex between a wild-type p66 subunit and a wild-type p55 subunit by the compound. The enhancement of complex formation between the p66 subunit having at least one mutation associated with an increase in resistance of HIV-1 to at least one NNRTI and the wild-type p55 subunit by the compound of the present invention is greater than 25% of the enhancement of complex formation between wild-type p66 subunit and wild-type p55 subunit using the compound, preferably at least 30% of the enhancement of complex formation between wild-type p66 subunit and wild-type p55 subunit, more preferably at least about 30% to about 100%, about 40% to about 100%, or about 50% to about 100% of the enhancement of complex formation between the wild-type p66 and the wild-type p66 subunit. In one embodiment, the compound provides enhancement of complex formation between the p66 subunit having at least one mutation associated with an increase in resistance of HIV-1 to at least one NNRTI and wild-type p55 subunit which is at least 50% of the enhancement of complex formation between the wild-type p66 subunit and wild-type p55 subunit. In another embodiment, the compound provides enhancement of complex formation between the p66 subunit having at least one mutation associated with an increase in resistance of HIV-1 to at least one NNRTI and wild-type p55 subunit which is at least 75% of the enhancement of complex formation between the wild-type p66 subunit and wild-type p55 subunit.

In another embodiment, the enhancement of formation of complex between the p66 subunit having at least one mutation associated with an increase in resistance of HIV-1 to at least one NNRTI and a wild-type p55 subunit by the compound of the present invention is about 100% to about 1000%; about 150% to about 1000%; preferably about 200% to about 1000%, more preferably about 500% to about 1000% or more of the enhancement of complex formation between wild-type p66 and wild-type p55 submit.

In another embodiment of the present invention, the compound provides higher enhancement of complex formation between a p66 subunit having at least one or more mutations associated with resistance of HIV-1 to at least one NNRTI and the wild-type p55 subunit compared to the enhancement of complex formation in the presence of a known NNRTI at a given concentration, that concentration being in a linear range of enhancement for both the compound and the known NNRTI. One such compound provides enhancement of complex formation between the p66 subunit having the single mutation and the wild-type p55 subunit which is higher than the enhancement in complex formation in the presence of (S)6-chloro-4(cyclopropylethynyl)-1,4-dihydro-4-(trifluoromethyl)-2H-3,1-benzoxazin-2-one (efavirenz) at a given concentration, that concentration being in a linear range of enhancement for both the compound and efavirenz. The enhancement by the compound of the present invention provides enhancement of complex formation which is at least 20% higher, preferably at least 25% higher, more preferably at least 30% to about 100% higher than the enhancement in complex formation in the presence of efavirenz at a given concentration, that concentration being in a linear range of enhancement for both the compound and efavirenz. The enhancement by the compound may provide enhancement which is 100%, 150%, 200%, 300%, 400%, 500% or as high as 1000% or more of the enhancement of complex formation in the presence of efavirenz at a given concentration, that concentration being in a linear range of enhancement for both the compound and efavirenz.

The present invention also provides a method of enhancing formation of a complex between a wild-type p66 subunit or a p66 subunit polypeptide having at least one mutation associated with resistance of HIV-1 reverse transcriptase and a wild-type p51 subunit or a p51 subunit polypeptide having the corresponding mutation as the p66 subunit of reverse transcriptase, which comprises contacting the subunits with an effective amount of at least one compound of the present invention so to thereby enhance formation of a complex between the wild-type p66 subunit or the p66 subunit polypeptide having at least one mutation and the wild-type p51 subunit or the p51 subunit polypeptide having the corresponding mutation as the p66 subunit of HIV-1 reverse transcriptase. In one aspect of the method, the HIV-1 reverse transcriptase is present in a subject and the contacting is effected by administering the compound to the subject.

The present invention provides a method of inhibiting the growth of HIV-1 comprising administration of at least one compound of the present invention in an amount effective to inhibit HIV-1. The HIV-1 may be wild-type HIV-1 or HIV-1 having one or more mutations associated with resistance to one or more NNRTIs. The method is particularly useful in inhibiting HIV-1 having one or more mutations in the reverse transcriptase subunit polypeptide(s), the mutation(s) associated with resistance to one or more NNRTIs, especially in subject in which the HIV-1 viral load has increased despite conventional NNRTI therapy. The method may further comprise the administration of one or more additional antiviral agents comprising one or more known NNRTIs, a nucleoside reverse transcriptase inhibitors, an HIV-1 protease inhibitors, or combinations thereof. Each additional anti-viral agent may be administered before or after administration of the compound of the present invention or co-administered with the compound of the present invention.

The present invention further provides a method of determining whether a compound enhances formation of a complex between a wild-type p66 subunit polypeptide of HIV-1 reverse transcriptase and a wild-type p51 subunit polypeptide of HIV-1 reverse transcriptase and/or enhances formation of a complex between a p66 subunit having at least one mutation associated with resistance of HIV-1 to at least one nonnucleoside reverse transcriptase inhibitor (NNRTI) and a wild-type p51 subunit of HIV-1 reverse transcriptase, said method comprises:

a) contacting a yeast cell with the compound, which cell comprises (i) a first plasmid which expresses a fusion protein comprising a wild-type p66 subunit polypeptide of HIV-1 reverse transcriptase, (ii) a second plasmid which expresses a fusion protein comprising a wild-type p51 subunit polypeptide of HIV-1 reverse transcriptase, and (iii) a reporter gene which is activated in the presence of a complex between the wild-type p66 subunit polypeptide and the wild-type p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the wild-type p51 subunit polypeptide of HIV-1 reverse transcriptase and the wild-type p66 subunit polypeptide of HIV-1 reverse transcriptase; and

c) contacting a yeast cell with the compound, which cell comprises (i) a third plasmid which expresses a fusion protein comprising a p66 subunit polypeptide of HIV-1 reverse transcriptase having at least one mutation associated with resistance of HIV-1 to at least one NNRTI, (ii) a second plasmid which expresses a fusion protein comprising a wild-type p51 subunit polypeptide of HIV-1 reverse transcriptase, and (iii) a reporter gene which is activated in the presence of a complex between the p66 subunit polypeptide having at least one mutation and the wild-type p51 subunit polypeptide, and determining the level of activity of the reporter gene in the cell in the presence of the compound; and

d) comparing the level of activity of the reporter gene determined in step (c) with a level of activity of the reporter gene determined in the absence of the compound, wherein an increased level of activity of the reporter gene determined in step (c) indicates that the compound is an activator of the formation of the complex between the wild-type p51 subunit polypeptide of HIV-1 reverse transcriptase and the p66 subunit polypeptide of HIV-1 reverse transcriptase having at least one mutation associated with resistance of HIV-1 to at least one NNRTI.

In the method, the mutation is associated with at least 5fold or greater resistance of HIV-1 to at least one NNRTI, preferably at least a ten-fold or greater resistance of HIV-1 to at least one NNRTI.

The above-described method of screening for compounds uses a third plasmid which expresses a fusion protein comprising a p66 subunit polypeptide of HIV-1 reverse transcriptase having at least one mutation associated with resistance of HIV-1 to at least one NNRTI and a second plasmid which expresses a fusion protein comprising a wild-type p51 subunit polypeptide of HIV-1 reverse transcriptase for optimal performance of the assay as it has been previously shown that NNRTIs bind tightly to p66 but not p51. However, modifications of the screening method may be made in which the p51 subunit having the mutations corresponding to those present in the p66 subunit polypeptide of reverse transcriptase is substituted for wild-type p51 in step (c) and step (d).

In conducting the method of determining whether a compound enhances formation of a complex, the p66 subunit polypeptide has one, two, three, four, five or more mutations, each mutation is associated with an increase in resistance of HIV-1 to at least one NNRTI. In one embodiment of the method, the p66 subunit polypeptide mutation is F227L, G190A, G190E, G190S, K101E, K103N, K238T, L100I, P225H, V106A, V106I, V108I, Y181C, Y188H, Y188L, or combinations thereof. In another embodiment of the method, the p66 subunit polypeptide has two mutations including but not limited to L100I and K103N; K103N and Y181C; K101E and K103N; K103N and Y188H; V106I and Y188L, K103N and P225H, K103N and V108I, and the like. In another embodiment of the method, the p66 subunit has a double mutation wherein the first mutation is K103N or Y188L and the second mutation is selected from the group consisting of V106I, V108I, Y181C, Y188H, P225H, and F227L.

The present invention further provides a method of determining whether a compound is capable of inhibiting wild-type HIV-1 reverse transcriptase activity and/or mutant HIV-1 reverse transcriptase activity, said method comprises:

a) contacting a yeast cell with the compound, which cell comprises (i) a first plasmid which expresses a fusion protein comprising a wild-type p66 subunit polypeptide of HIV-1 reverse transcriptase, (ii) a second plasmid which expresses a fusion protein comprising a wild-type p51 subunit polypeptide of HIV-1 reverse transcriptase, and (iii) a reporter gene which is activated in the presence of a complex between the wild-type p66 subunit polypeptide and the wild-type p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the wild-type p51 subunit polypeptide of HIV-1 reverse transcriptase and the wild-type p66 subunit polypeptide of HIV-1 reverse transcriptase, thereby indicating that the compound is capable of inhibiting wild-type HIV-1 reverse transcriptase activity; and

c) contacting a yeast cell with the compound, which cell comprises (i) a third plasmid which expresses a fusion protein comprising a p66 subunit polypeptide of HIV-1 reverse transcriptase having at least one mutation associated with resistance of HIV-1 to at least one NNRTI, (ii) a second plasmid which expresses a fusion protein comprising a wild-type p51 subunit polypeptide of HIV-1 reverse transcriptase, and (iii) a reporter gene which is activated in the presence of a complex between the p66 subunit polypeptide having at least one mutation and the wild-type p51 subunit polypeptide, and determining the level of activity of the reporter gene in the cell in the presence of the compound; and

d) comparing the level of activity of the reporter gene determined in step (c) with a level of activity of the reporter gene determined in the absence of the compound, wherein an increased level of activity of the reporter gene determined in step (c) indicates that the compound is an activator of the formation of the complex between the wild-type p51 subunit polypeptide of HIV-1 reverse transcriptase and the p66 subunit polypeptide of HIV-1 reverse transcriptase having at least one mutation associated with resistance of HIV-1 to at least one NNRTI, thereby indicating that the compound is capable of inhibiting mutant HIV-1 reverse transcriptase activity.

This invention provides a method of determining whether a compound inhibits wild-type or mutant HIV-1 reverse transcriptase which comprises:

a) contacting a yeast cell with the compound, which cell comprises (i) a first plasmid which expresses a fusion protein comprising a p66 subunit polypeptide of wild-type or mutant HIV-1 reverse transcriptase, (ii) a second plasmid which expresses a fusion protein comprising a p51 subunit polypeptide of HIV-1 reverse transcriptase, and (iii) a reporter gene which is activated in the presence of a complex between the p66 subunit polypeptide and the p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the p51 subunit polypeptide of HIV-1 reverse transcriptase and the p66 subunit polypeptide of wild-type or mutant HIV-1 reverse transcriptase, thereby indicating that the compound inhibits wild-type or mutant HIV-1 reverse transcriptase.

This invention provides a method of determining whether a compound enhances formation of a complex between a p66 subunit polypeptide of wild-type or mutant HIV-1 reverse transcriptase and a p51 subunit polypeptide of HIV-1 reverse transcriptase which comprises:

a) contacting a yeast cell with the compound, which cell comprises (i) a first plasmid which expresses a fusion protein comprising a p66 subunit polypeptide of wild-type or mutant HIV-1 reverse transcriptase, (ii) a second plasmid which expresses a fusion protein comprising a p51 subunit polypeptide of HIV-1 reverse transcriptase, and (iii) a reporter gene which is activated in the presence of a complex between the p66 subunit polypeptide and the p51 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the p51 subunit polypeptide of HIV-1 reverse transcriptase and the p66 subunit polypeptide of wild-type or mutant HIV-1 reverse transcriptase.

This invention provides a method of determining whether a compound inhibits HIV-1 reverse transcriptase which comprises:

a) contacting a yeast cell with the compound, which cell comprises (i) a first plasmid which expresses a fusion protein comprising a first p66 subunit polypeptide of HIV-1 reverse transcriptase, (ii) a second plasmid which expresses a fusion protein comprising a second p66 subunit polypeptide of HIV-1 reverse transcriptase, and (iii) a reporter gene which is activated in the presence of a complex between the first p66 subunit polypeptide and the second p66 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene in step (a) indicates that the compound is an activator of the formation of the complex between the first p66 subunit polypeptide of HIV-1 reverse transcriptase and the second p66 subunit polypeptide of HIV-1 reverse transcriptase, thereby indicating that the compound inhibits HIV-1 reverse transcriptase.

This invention provides a method of determining whether a compound enhances formation of a complex between a first p66 subunit polypeptide of HIV-1 reverse transcriptase and a second p66 subunit polypeptide of HIV-1 reverse transcriptase which comprises:

a) contacting a yeast cell with the compound, which cell comprises (i) a first plasmid which expresses a fusion protein comprising a first p66 subunit polypeptide of HIV-1 reverse transcriptase, (ii) a second plasmid which expresses a fusion protein comprising a second p66 subunit polypeptide of HIV-1 reverse transcriptase, and (iii) a reporter gene which is activated in the presence of a complex between the first p66 subunit polypeptide and the second p66 subunit polypeptide, and determining the level of activity of the reporter gene in the cell in the presence of the compound; and

b) comparing the level of activity of the reporter gene determined in step (a) with a level of activity of the reporter gene determined in the absence of the compound, wherein an increased level of activity of the reporter gene in step (a) indicates that the compound is an activator of the formation of the complex between the first p66 subunit polypeptide of HIV-1 reverse transcriptase and the second p66 subunit polypeptide of HIV-1 reverse transcriptase, thereby indicating that the compound inhibits HIV-1 reverse transcriptase.

The methods described herein may also be adapted to other types of cells in addition to a yeast cell. Other cell types include but are not limited to eucaryotic, procaryotic, bacteria, E. coli, mammalian and human cells.

In one embodiment of the methods described herein, (a) the fusion protein expressed by the first or third plasmid comprises a peptide having a DNA binding domain, and (b) the fusion protein expressed by the second plasmid comprises a peptide having a transcription activation domain.

In one embodiment of the methods described herein, (a) the fusion protein expressed by the first or third plasmid comprises a peptide having a transcription activation domain, and (b) the fusion protein expressed by the second plasmid comprises a peptide having a DNA binding domain.

In one embodiment of the fusion proteins described herein, the peptide having a DNA binding domain is N-terminal relative to the p66 or p51 subunit polypeptide. In another embodiment, the peptide having a DNA binding domain is C-terminal relative to the 51 or p66 subunit polypeptide. The peptide having a DNA binding domain may be bound in the fusion protein to the p51 or p66 subunit polypeptides. In one embodiment, they are bound by peptide bonds. Alternatively, the fusion protein may also comprise one or more additional components, such as a peptide linker and/or an epitope tag. These additional components may separate the peptides from the p51 or p66 subunit polypeptide. The various components may be bound to each other by peptide bonds. In one preferred embodiment, the p66 subunit polypeptide is from derived from p66SH2-1 and the p51 subunit is derived from p51GADNOT for use in the method of screening for compounds of the present invention.

In one embodiment of the fusion proteins described herein, the peptide having a transcription activation domain is N-terminal relative to the p66 or p51 subunit polypeptide. In another embodiment, the peptide having a transcription activation domain is C-terminal relative to the 51 or p66 subunit polypeptide. The peptide having a transcription activation domain may be bound in the fusion protein to the p51 or p66 subunit polypeptides. In one embodiment, they are bound by peptide bonds. Alternatively, the fusion protein may also comprise one or more additional components, such as a peptide linker and/or an epitope tag. These additional components may separate the peptides from the p51 or p66 subunit polypeptide. The various components may be bound to each other by peptide bonds.

The invention described herein may employ p51 and p66 subunits from among various HIV-1 strains. One may use the reverse transcriptase coding regions for p66 and p51 from any HIV-1 strain. For example, in the HIV-1 NL4-3 strain, the amino acid and nucleic acid sequences of which may be found in a pNL4-3 clone deposited at Genbank Accession No. M19921. The p51 and p66 subunits share the same N-terminal sequence, whereas p51 does not have the C-terminal ribonuclease H region. Accordingly, p66 corresponds to codons 1-560 and p51 corresponds to codons 1-440 in the RT gene.

The invention may employ other HIV-1 strains such as the following: HIV_{HXB2G} (Genbank Accession No. K03455), HIV_{BRUCG} (Genbank Accession No. K02013), HIV_{MNCG} (Genbank Accession No. M17449), HIV_{NY5CG} (Genbank Accession No. M38431); HIV_{JRCCSF} (Genbank Accession No.M38429), and HIV_{SF2CG} (Genbank Accession No. K02007).

The invention described herein may employ p66 subunits of HIV-1 reverse transcriptase having one or more mutations. The one or more mutations in the p66 subunit are associated with increased drug resistance of HIV-1. Of particular importance are mutations in the p66 subunit associated with increased drug resistance of clinical isolates of HIV-1, in particular clinical isolates of HIV-1 resistant to one or more NNRTIs. Preferably the mutation(s) in the p66 subunit of HIV-1 reverse transcriptase is (are) associated with resistance of HIV-1 to at least one or more NNRTI, in particular, NNRTIs known in the art. In one embodiment, the p66 subunit polypeptide mutation is F227L, G190A, G190E, G190S, K101E, K103N, K238T, L1001, P225H, V106A, V106I, V108I, Y181C, Y188H, Y188L, and combinations thereof. In another embodiment, the p66 subunit has two, three, four, five or more mutations, each mutation associated with an increase in resistance of HIV-1 to at least one NNRTI.

Non-limiting examples of double mutations in the p66 subunit polypeptide of HIV-1 reverse transcriptase include but are not limited to L1001 and K103N; K103N and Y181C; K101E and K103N; K103N and Y188H; V106I and Y188L; K103N and P225H, K103N and V108I, and the like. Such mutations have been reported to be associated with resistance to at least one NNRTI (Bacheler, L. et al, ibid).

One may use the reverse transcriptase coding region for p66 from any HIV-1 strain, provided the p66 has at least one mutation associated with resistance to at least one NNRTI. Such strains of HIV-1 may be clinical isolates taken from subjects, or the mutation may be induced in vitro by treatment of the HIV-1 with at least one NNRTI. One or more mutations in p66 may also be introduced into a wild-type coding region of p66 by site-directed mutagenesis using methods known in the art (Bacheler, L. et al, ibid, Tachedjian, G et al, 2001 ibid).

In one embodiment of the fusion proteins described herein, the DNA binding domain is a LexA DNA binding domain. The amino acid and nucleic acid sequences for LexA may be found at Genbank Accession No. J01643. In one embodiment of the methods described herein, the peptide having a DNA binding domain comprises LexA amino acid residues 1-87. The portion of LexA which corresponds to amino acid residues 1-87 may comprise a LexA DNA binding domain. In one embodiment of the methods described herein, the peptide having a DNA binding domain comprises LexA amino acid residues 1-202. The portion of LexA which corresponds to amino acid residues 1-202 may comprise a LexA DNA binding domain.

In one embodiment of the fusion proteins described herein, the DNA binding domain is a GAL4 DNA binding domain. The amino acid and nucleic acid sequences for Gal4 may be found at Genbank Accession No. K01486.

In one embodiment of the fusion proteins described herein, the transcription activation domain is a GAL4 transcription activation domain. In one embodiment, the peptide having a transcription activation domain comprises GAL4 amino acid residues 768-881. The portion of Gal4 which corresponds to amino acid residues 768-881 may comprise a Gal4 activation domain.

In one embodiment of the fusion proteins described herein, the transcription activation domain is a VP16 transcription activation domain. The amino acid and nucleic acid sequences for VP16 may be found at Genbank Accession No. U89963.

In one embodiment of the fusion proteins described herein, the fusion protein expressed by the first and third plasmid, the second plasmid or each plasmid comprises a peptide comprising consecutive alanine residues. The above described peptide comprising consecutive alanine residues may be referred to as an alanine linker. Such linker sequence may be a series of consecutive amino acid residues other than alanine. Such linker sequence may be of various lengths. For example, the linker may comprise 2 amino acids, 3 amino acids, 4 amino acids, 5 amino acids, 6 amino acids, 7 amino acids, 8 amino acids, 9 amino acids or 10 amino acids. The peptide linker may also be of longer lengths, for example, from about 10 amino acids to about 20 amino acids. In one embodiment, the peptide comprising consecutive alanine residues comprises at least 6 alanine residues.

As used herein, the following standard abbreviations are used throughout the specification to indicate specific amino acids: A=ala=alanine; R=arg=arginine; N=asn=asparagine; D=asp=aspartic acid; C=cys=cysteine; Q=gln=glutamine; E=glu=glutamic acid; G=gly=glycine; H=his=histidine; I=ile=isoleucine; L=leu=leucine; K=lys=lysine; M=met=methionine; F=phe=phenylalanine; P=pro=proline; S=ser=serine; T=thr=threonine; W=trp=tryptophan; Y=tyr=tyrosine; V=val=valine; B=asx=asparagine or aspartic acid; Z=glx=glutamine or glutamic acid.

As used herein, the following standard abbreviations are used throughout the specification to indicate specific nucleotides: C=cytosine; A=adenosine; T=thymidine; G=guanosine; and U=uracil.

In one embodiment of the fusion proteins described herein, the fusion protein comprises an influenza hemagglutinin (HA) epitope tag. The sequence for influenza hemagglutinin (HA) epitope may be found in Genbank Accession No. U29899 at nucleotide bases 5042-5068 within the plasmid pACT2. The invention may also comprise other types of epitope tags known to one skilled in the art.

In one embodiment of the fusion proteins described herein, the reporter gene is a LacZ reporter gene. The amino acid and nucleic acid sequences for LacZ may be found at Genbank Accession no. U89671.

In one embodiment of the methods described herein, (a) the fusion protein expressed by the first plasmid comprises a peptide comprising a LexA protein DNA binding domain, wherein the p66 subunit polypeptide is bound at its C-terminal amino acid to the N-terminal amino acid of the peptide comprising a LexA protein DNA binding domain; and (b) the fusion protein expressed by the second plasmid comprises a Gal4 peptide corresponding to amino acids 768-881 of Gal4, and an influenza hemagglutinin (HA) epitope tag, which Gal4 peptide is bound at its C-terminal amino acid to the N-terminal amino acid of the influenza hemagglutinin (HA) epitope tag, which influenza hemagglutinin (HA) epitope tag is bound at its C-terminal amino acid to the N-terminal amino acid of the p51 subunit polypeptide.

In the fusion proteins described herein, the location of the various components relative to each other may be varied. For example, in the embodiment described above, the peptide comprising a LexA protein DNA binding domain may alternatively be bound at its C-terminal amino acid to the N-terminal amino acid of the p66 subunit polypeptide. The p51 subunit polypeptide may be N-terminal to the Gal4 peptide. One skilled in art would know how to make and use the various vectors and plasmids described herein.

In one embodiment of the methods described herein, (a) the fusion protein expressed by the first or third plasmid comprises a peptide comprising a LexA protein DNA binding domain, wherein the p66 subunit polypeptide is bound at it's C-terminal amino acid to the N-terminal amino acid of the peptide comprising a LexA protein DNA binding domain; and (b) the fusion protein expressed by the second plasmid comprises a Gal4 peptide corresponding to amino acids 768-881 of Gal4, which Gal4 peptide is bound at its C-terminal amino acid to the N-terminal amino acid of the p51 subunit polypeptide.

In one embodiment of the methods described herein, (a) the fusion protein expressed by the first or third plasmid comprises a LexA peptide corresponding to amino acid residues 1-87, wherein the LexA peptide is bound at its C-terminal amino acid to the N-terminal amino acid of the of the p66 subunit polypeptide; and (b) the fusion protein expressed by the second plasmid comprises a Gal4 peptide corresponding to amino acids 768-881 of Gal4, and an influenza hemagglutinin (HA) epitope tag, which Gal4 peptide is bound at its C-terminal amino acid to the N-terminal amino acid of the influenza hemagglutinin (HA) epitope tag, which influenza hemagglutinin (HA) epitope tag is bound at its C-terminal amino acid to the N-terminal amino acid of the p51 subunit polypeptide.

In one embodiment of the methods described herein, (a) the fusion protein expressed by the first or third plasmid comprises a LexA peptide corresponding to amino acid residues 1-87, wherein the LexA peptide is bound at its C-terminal amino acid to the N-terminal amino acid of the of the p66 subunit polypeptide; and (b) the fusion protein expressed by the second plasmid comprises a Gal4 peptide corresponding to amino acids 768-881 of Gal4, which Gal4 peptide is bound at its C-terminal amino acid to the N-terminal amino acid of the p51 subunit polypeptide.

In one embodiment of the methods described herein, (a) the fusion protein expressed by the first or third plasmid comprises a LexA peptide corresponding to amino acid residues 1-202, and a peptide comprising six consecutive alanine residues, wherein the LexA peptide is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide; and (b) the fusion protein expressed by the second plasmid comprises a Gal4 peptide corresponding to amino acids 768-881 of Gal4, which Gal4 peptide is bound at its C-terminal amino acid to the N-terminal amino acid of the p51 subunit polypeptide.

In one embodiment of the methods described herein, (a) the fusion protein expressed by the first or third plasmid comprises a LexA peptide corresponding to amino acid residues 1-202, and a peptide comprising six consecutive alanine residues, wherein the LexA peptide is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide; and (b) the fusion protein expressed by the second plasmid comprises a Gal4 peptide corresponding to amino acids 768-881 of Gal4, and an influenza hemagglutinin (HA) epitope tag, which Gal4 peptide is bound at its C-terminal amino acid to the N-terminal amino acid of the influenza hemagglutinin (HA) epitope tag, which influenza hemagglutinin (HA) epitope tag is bound at its C-terminal amino acid to the N-terminal amino acid of the p51 subunit polypeptide.

In one embodiment of the methods described herein, (a) the fusion protein expressed by the first or third plasmid comprises a Gal4 peptide corresponding to amino acids 768-881 of Gal4, an influenza hemagglutinin (HA) epitope tag, and a peptide comprising six consecutive alanine residues, wherein the Gal4 peptide is bound at its C-terminal amino acid to the N-terminal amino acid of the influenza hemagglutinin (HA) epitope tag, wherein the influenza hemagglutinin (HA) epitope tag is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide; and (b) the fusion protein expressed by second plasmid comprises a peptide comprising a LexA protein DNA binding domain, wherein the p51 subunit polypeptide is bound at its C-terminal amino acid to the N-terminal amino acid of the peptide comprising a LexA protein DNA binding domain.

In one embodiment of the methods described herein, (a) the fusion protein expressed by the first or third plasmid comprises a Gal4 peptide corresponding to amino acids 768-881 of Gal4, an influenza hemagglutinin (HA) epitope tag, and a peptide comprising six consecutive alanine residues, wherein the Gal4 peptide is bound at its C-terminal amino acid to the N-terminal amino acid of the influenza hemagglutinin (HA) epitope tag, wherein the influenza hemagglutinin (HA) epitope tag is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide; and (b) the fusion protein expressed by second plasmid comprises a peptide comprising a LexA protein DNA binding domain, wherein peptide comprising a LexA protein DNA binding domain is bound at its C-terminal amino acid to the N-terminal amino acid of the p51 subunit polypeptide.

In one embodiment of the methods described herein, (a) the fusion protein expressed by the first or third plasmid comprises a Gal4 peptide corresponding to amino acids 768-881 of Gal4, an influenza hemagglutinin (HA) epitope tag, and a peptide comprising six consecutive alanine residues, wherein the Gal4 peptide is bound at its C-terminal amino acid to the N-terminal amino acid of the influenza hemagglutinin (HA) epitope tag, wherein the influenza hemagglutinin (HA) epitope tag is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide; and (b) the fusion protein expressed by second plasmid comprises a peptide comprising a Gal4 protein DNA binding domain, which peptide comprising a Gal4 protein DNA binding domain is bound at its C-terminal amino acid to the N-terminal amino acid of the p51 subunit polypeptide.

In one embodiment of the methods described herein, (a) the fusion protein expressed by the first or third plasmid comprises a Gal4 peptide corresponding to amino acids 768-881 of Gal4, wherein the Gal4 peptide is bound at its C-terminal amino acid to the N-terminal amino acid of the p66 subunit polypeptide; and (b) the fusion protein expressed by second plasmid comprises a peptide comprising a LexA protein DNA binding domain, wherein the p51 subunit polypeptide is bound at its C-terminal amino acid to the N-terminal amino acid of the peptide comprising a LexA protein DNA binding domain.

In one embodiment of the methods described herein, (a) the fusion protein expressed by the first or third plasmid comprises a Gal4 peptide corresponding to amino acids 768-881 of Gal4, wherein the Gal4 peptide is bound at its C-terminal amino acid to the N-terminal amino acid of the p66 subunit polypeptide; and (b) the fusion protein expressed by second plasmid comprises a peptide comprising a LexA protein DNA binding domain, which peptide comprising a LexA protein DNA binding domain is bound at its C-terminal amino acid to the N-terminal amino acid of the p51 subunit polypeptide.

In one embodiment of the methods described herein, (a) the fusion protein expressed by the first or third plasmid comprises a Gal4 peptide corresponding to amino acids 768-881 of Gal4, wherein the Gal4 peptide is bound at its C-terminal amino acid to the N-terminal amino acid of the p66 subunit polypeptide; and (b) the fusion protein expressed by second plasmid comprises a peptide comprising a Gal4 protein DNA binding domain, which peptide comprising a Gal4 protein DNA binding domain is bound at its C-terminal amino acid to the N-terminal amino acid of the p51 subunit polypeptide.

In one embodiment of the methods described herein, (a) the fusion protein expressed by the first or third plasmid comprises a peptide comprising a LexA protein DNA binding domain, wherein the p66 subunit polypeptide is bound at it's C-terminal amino acid to the N-terminal amino acid of the peptide comprising a LexA protein DNA binding domain; and (b) the fusion protein expressed by the second plasmid comprises a Gal4 peptide corresponding to amino acids 768-881 of Gal4, an influenza hemagglutinin (HA) epitope tag, and a peptide comprising six consecutive alanine residues, wherein the Gal4 peptide is bound at its C-terminal amino acid to the N-terminal amino acid of the influenza hemagglutinin (HA) epitope tag, wherein the influenza hemagglutinin (HA) epitope tag is bound at its C-terminal amino acid to the N-terminal amino acid of the peptide comprising six consecutive alanine residues, wherein the peptide comprising six consecutive alanine residues is bound at its C-terminal amino acid to the N-terminal amino acid of the p66 subunit polypeptide.

This invention provides a method of making a pharmaceutical composition which comprises:

a) determining whether a compound inhibits HIV-1 reverse transcriptase by one of the methods described herein;

b) recovering the compound ; and

c) admixing the compound with a pharmaceutically acceptable carrier.

As used herein, “inhibits” as used in the screening methods means that the amount of signal from a reporter gene in the Y2H system is reduced as compared with the amount that would occur in a control sample without the compound. As used herein “enhanced” as used in the screening methods means that the amount of signal from a reporter gene in the Y2H system is increased compared with the amount that would occur in a control sample without the compound. As used herein, “inhibits” also means that the amount of growth of HIV-1 when grown in vitro in cultured cells or in vivo infected cells is reduced as compared with the amount that would occur in a control sample without the compound.

As used herein, the term “compound” includes both protein and non-protein moieties. In one embodiment, the compound is a small molecule. In another embodiment, the compound is a protein. The protein may be, by way of example, an antibody directed against a portion of a p51 or p66 subunit. The agent may be derived from a library of low molecular weight compounds or a library of extracts from plants or other organisms. Combinatorial chemical libraries as well as traditional, synthetic chemical libraries are available in the art such as those from ChemBridge Corp. (San Diego, Calif.). The novel compounds of the present invention are not previously known or are not previously known to be capable of enhancing the interaction of p66 and p51, in particular, not previously known to be capable of enhancing interaction of these subunits when p66 carries one or more mutations associated with resistance to an NNRTI. The compounds of the present invention may be a derivative of a known compound, in particular a derivative of a known NNRTI. The compounds of the present invention may be a derivative of efavirenz, UC-781, HBY 087, Nevirapine, delavirdine, SJ-3366, MKC-442, GW420867x, HI-443, and the like. The agents of the subject invention include but are not limited to compounds or molecular entities such as peptides, polypeptides, and other organic or inorganic molecules and combinations thereof.

In one embodiment of the methods described herein, the compound is a polypeptide. In one embodiment of the methods described herein, the compound is a oligopeptide. In one embodiment of the methods described herein, the compound is a nonpeptidyl agent. In one embodiment, the nonpeptidyl agent is a compound having a molecular weight less than 500 daltons. In another embodiment the compound is a small chemical molecule having a molecular weight of 1000 daltons or less.

In addition to the primary method of determining whether a compound enhances formation of a complex between a p66 subunit having at least one mutation associated with resistance of HIV-1 reverse transcriptase, compounds identified by the method herein, may be screened for additional beneficial characteristics as a drug candidate for HIV-1 therapy using methods known in the art. Positive compounds for enhancement of complex formation may be tested in a viral growth assay. Compounds of the present invention have IC₅₀ values against growth of the HIV-1 virus in the range of about 0.01 to about 20,000 nM, preferably IC₅₀ values less than one nanomolar, more preferably IC₅₀ values in the range of about 0.01 to about 1 nM.

The compounds of the present invention may have other advantages over prior art compounds, in particular, advantages over prior art NNRTIs, in addition to the benefit of being effective against HIV-1 having at least one or more mutations in the p66 subunit polypeptide of reverse transcriptase. Compounds of the present invention may additionally be selected for those compounds that have fewer side effects that those associated with known NNRTI therapy currently in clinical use. Such side effects which are desirable to be avoided include but are not limited to severe rash, skin reactions, psychiatric symptoms, nervous system symptoms, and the like.

In one embodiment of the methods of using the compound of the present invention described herein, the reverse HIV-1 transcriptase enzyme or its p51 and p66 subunits is present in a subject and the contacting is effected by administering the compound to the subject. Accordingly, the subject invention has various applications which includes HIV treatment such as treating a subject who has become afflicted with HIV. As used herein, “afflicted with HIV” means that the subject has at least one cell which has been infected by HIV. As used herein, “treating” means either slowing, stopping or reversing the progression of an HIV disorder. In the preferred embodiment, “treating” means reversing the progression to the point of eliminating the disorder. As used herein, “treating” also means the reduction of the number of viral infections, reduction of the number of infectious viral particles, reduction of the number of virally infected cells, or the amelioration of symptoms associated with HIV. Another application of the subject invention is to prevent a subject from contracting HIV. As used herein, “contracting HIV” means becoming infected with HIV, whose genetic information replicates in and/or incorporates into the host cells. Another application of the subject invention is to treat a subject who has become infected with HIV. As used herein, “HIV infection” means the introduction of HIV genetic information into a target cell, such as by fusion of the target cell membrane with HIV or an HIV envelope glycoprotein⁺ cell. The target cell may be a bodily cell of a subject. In the preferred embodiment, the target cell is a bodily cell from a human subject. Another application of the subject invention is to inhibit HIV infection. As used herein, “inhibiting HIV infection” means reducing the amount of HIV genetic information introduced into a target cell population as compared to the amount that would be introduced without said composition.

This invention provides a method of treating a subject afflicted with HIV which comprises administering to the subject an effective dose of an agent of composition described herein. In one embodiment, the agent or composition may be enough to decrease the subject's viral load. As used herein, “treating” means either slowing, stopping or reversing the progression of an HIV disorder. In the preferred embodiment, “treating” means reversing the progression to the point of eliminating the disorder. As used herein, “treating” also means the reduction of the number of viral infections, reduction of the number of infectious viral particles, reduction of the number of virally infected cells, or the amelioration of symptoms associated with HIV such as in AIDS and ARC (AIDS related complex). As used herein, “afflicted with HIV” means that the subject has at least one cell which has been infected by HIV.

This invention provides a method of preventing a subject from contracting HIV which comprises administering to the subject an effective dose of an agent or composition described herein.

The dose of the agent or composition of the invention will vary depending on the subject and upon the particular route of administration used. Dosages can range from 0.1 to 100,000 μg/kg. Based upon the composition, the dose can be delivered continuously, such as by continuous pump, or at periodic intervals. For example, on one or more separate occasions. Desired time intervals of multiple doses of a particular composition can be determined without undue experimentation by one skilled in the art.

As used herein, “effective dose” means an amount in sufficient quantities to either treat the subject or prevent the subject from becoming HIV infected. A person of ordinary skill in the art can perform simple titration experiments to determine what amount is required to treat the subject. As used herein, “contracting HIV” means becoming infected with HIV, whose genetic information replicates in and/or incorporates into the host cells. In one embodiment, the effective amount of the agent or composition comprises from about 0.000001 mg/kg body weight to about 100 mg/kg body weight of the subject.

As used herein, “subject” means any animal or artificially modified animal capable of becoming HIV-infected. The subjects include but are mot limited to a human being, a non-human primate, an equine, an opine, an avian, a bovine, a porcine, a canine, a feline or a mouse. Artificially modified animals include, but are not limited to, SCID mice with human immune systems. The animals include but are not limited to mice, rats, dogs, guinea pigs, ferrets, rabbits, and primates. In the preferred embodiment, the subject is a human being. The subject may be an “HIV-infected subject” which is a subject having at least one of his or her own cells invaded by HIV. In the preferred embodiment, the HIV infected subject is a human being. The subject may be a “non-HIV-infected subject” which is a subject not having any of his own cells invaded by HIV. In the preferred embodiment, the non-HIV infected subject is a human being.

As used herein, “administering” may be effected or performed using any of the methods known to one skilled in the art, which includes intralesional, intraperitoneal, intramuscular, subcutaneous, intravenous, liposome mediated delivery, transmucosal, intestinal, topical, nasal, oral, anal, ocular, otic delivery, and the like.

In one embodiment, amount of the compound administered is between about 1 mg and about 50 mg per kg body weight of the subject. In one embodiment, amount of the compound administered is between about 2 mg and about 40 mg per kg body weight of the subject. In one embodiment, amount of the compound administered is between about 3 mg and about 30 mg per kg body weight of the subject. In one embodiment, the amount of the compound administered is between about 4 mg and about 20 mg per kg body weight of the subject. In one embodiment, amount of the compound administered is between about 5 mg and about 10 mg per kg body weight of the subject.

In one embodiment of the methods described herein, the compound is administered at least once per day. In one embodiment of the methods described herein, the agent is administered daily. In one embodiment of the methods described herein, the agent is administered every other day. In one embodiment of the methods described herein, the agent is administered every 6 to 8 days. In one embodiment of the methods described herein, the agent is administered weekly.

In one embodiment of the enhancement or treatment methods, the HIV-1 reverse transcriptase is present in a subject and the contacting is effected by administering the compound to the subject. The compound may be administered by various routes known to one skilled in the art including but not limited to those wherein the compound is administered orally, intravenously, subcutaneously, intramuscularly, topically or by liposome-mediated delivery. The subject may be any subject including but not limited to a human being, a non-human primate, an equine, an opine, an avian, a bovine, a porcine, a canine, a feline or a mouse. In one embodiment, the compound is administered at least once per day. In one embodiment, the compound is administered daily. In one embodiment, the compound is administered every other day. In one embodiment, compound is administered every 6 to 8 days. In one embodiment, the compound is administered weekly.

This invention provides a composition which comprises one of the compounds described herein and a carrier. As used herein, “composition” means a mixture. The compositions include but are not limited to those suitable for oral, rectal, intravaginal, topical, nasal, opthalmic, or parenteral, intravenous, subcutaneous, intramuscular, and intraperitoneal administration to a subject. As used herein, “parenteral” includes but is not limited to subcutaneous, intravenous, intramuscular, or intrasternal injections or infusion techniques.

This invention provides an agent or pharmaceutical composition described herein and a carrier. Such carrier may be one that is a pharmaceutically acceptable carrier. Pharmaceutically acceptable carriers are well known to those skilled in the art. Such pharmaceutically acceptable carriers may include but are not limited to aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases and the like.

The present invention further provides a pharmaceutical composition comprising one or more compounds capable of enhancing formation of a complex between a wild-type p66 and/or p66 subunit polypeptide of HIV-1 reverse transcriptase having at least one mutation associated with an increase in resistance to at least one NNRTI and a wild-type p51 subunit or mutant p51 subunit having a mutation corresponding to the mutation in the p66 subunit polypeptide of HIV-1 reverse transcriptase and a pharmaceutically acceptable carrier, in combination with one or more additional anti-viral agents selected from the group consisting of a known NNRTI, a nucleoside reverse transcriptase inhibitor, an HIV-1 protease inhibitor, and combinations thereof.

The known NNRTI compounds that may be used in the composition of the present invention include but are not limited to efavirenz, UC-781, HBY 097, nevirapine (11-cyclopropyl-5,11,-dihydro-4-methyl-6H-dipyrido[3,2-b:2′3′-][1,4] diazepin-6-one), delavirdine ((Rescriptor™; Pharmacia Upjohn) (piperazine, 1-[3-[(1-methyl-ethyl)amino]-2-pyridinyl]-4-[[5-[(methysulfonyl)amino]-1H-indol-2-yl]carbonyl]-, monomethanesulfonate), SJ-3366 (1-(3-cyclopenten-1-yl)methyl-6-(3,5-dimethylbenzoyl)-5-ethyl-2,4-pyrimidinedione), MKC-442 (6-benzyl-1-(ethoxymethyl)-5-isopropyluracil), GW420867x (S-3 ethyl-6-fluro-4-isopropoxycarbonyl-3,4-dihydro-quinoxalin-2(1H)-one; Glaxo), HI-443 (N′-[2-(2-thiophene)ethyl]-N′-[2-(5-bromopyridyl)]-thiourea), and the like.

The nucleoside reverse transcriptase inhibitors that may be used in the composition in combination with at least one novel compound of the present invention include but are not limited to abacavir (Ziagen™, GlaxoSmithKline) ((1S,cis)-4-[2-amino-6-(cyclopropylamino)-9H-purin-9-yl]-2-cyclopentene-1-methanol sulfate (salt)), lamivudine (Epivir™, GlaxoSmthKline) ((2R, cis)-4-amino-1-(2-hydroxymethyl-1,3-oxathiolan-5-yl)-(1H)-pyrimidin-2-one), zidovudine (Retrovir™; GlaxoSmithKline) (3′azido-3′-deoxythymidine), stavudine (Zerit; Bristol-Myers Squibb) (2′,3′-didehydro-3′deoxythymidine), zacitabine (Hivid™; Roche Laboratories) (4-amino-1-beta-D2′,3′-dideoxyribofuranosyl-2-(1H)-pyrimidone), didanosine, and the like.

The HIV-1 protease inhibitors that may be used in the composition in combination with a novel compound of the present invention include but are not limited to lopinavir (1S-[1R*,(R*),3R*,4R*]]-N-4-[[(2,6-dimethyphenoxy)acetyl]amino]-3-hydroxy-5-phenyl-1-(phenylmethyl)pentyl]tetrahydro-alpha-(1-methylethyl)-2-oxo1(2H)-pyrimidineacetamide), saquinavir (N-tert-butyl-decahydro-2-[2(R)-hydroxy-4-phenyl-3(S)-[[N-(2-quinolylcarbonyl)-L-asparaginyl]amino]butyl]-(4aS,8aS)-isoquinoline-(3S)-carboxamide), nelfinavir mesylate ([3S-[2(2S*,3S*),3a,4β,8aβ]]-N-(1,1-dimethyetyl)decahydro-2[2-hydroxy-3-[(3-hydroxy-2-methylbenzoyl)amino]-4-(phenylthio)butyl]-3-isoquinolinecarboxamide mono-methane sulfonate), indinavir sulfate (([1(1S,2R),5(S))]-2,3,5-trideoxy-N-(2,3-dihydro-2-hydroxy-1H-inden-1-yl)-5-[2-[[(1,1-dimethylethyl)amino]carbonyl]-4-(3-pyridinylmethyl)-1-piperazinyl]-2-(phenylmethyl)-D-erythropentonamide sulfate (1:1) salt), amprenavir ((3S)-tetrahydro-3-furyl N-[(1S,2R)-3-(4-amino-N-isobutylbenzenesulfonamido)-1-benzyl-2-hydroxypropyl]carbamate), ritonavir ((10-Hydroxy-2-methyl-5-(1-methylethyl)-1-[2-(1-methylethyl)-4-thiazolyl]-3,6-dioxo-8,11-bis (phenylmethyl)-2,4,7,12-tetraazatridecan-13-oic acid, 5-thiazolylmethyl ester, [5S-(5R*, 8R*, 10R*, 11R*)]), and the like.

In one embodiment of the agents described herein, the agent is a polypeptide. In one embodiment of the agents described herein, the agent is a oligopeptide. As used herein, “polypeptide” means two or more amino acids linked by a peptide bond.

The nucleic acids, polypeptides and antibodies described herein may be isolated and/or purified. One skilled in the art would know how to isolate and/or purify them. Methods are provided in any laboratory manual such as “Molecular Cloning” by Samrook, Fritsch and Maniatis.

In one embodiment of the agents described herein, the compound is a nonpeptidyl agent. As used herein, “nonpeptidyl agent” means an agent that does not consist in its entirety of a linear sequence of amino acids linked by peptide bonds. A nonpeptidyl molecule may, however, contain one or more peptide bonds. In one embodiment, the nonpeptidyl agent is a compound having a molecular weight less than 500 daltons. As used herein, a “small molecule” is one having a molecular weight less than 1000 daltons, and may be in a range of 1000 to 500 daltons or less.

The polypeptides described herein may be made by any means known to one skilled in the art. For example, the protein may be made by recombinant expression from a nucleic acid, such as a plasmid or vector comprising the encoding nucleic acid, wherein the plasmid or vector is in a suitable host cell, i.e. a host-vector system for the production of the polypeptide of interest. A suitable vector may be made which comprises suitable regulatory sequences, such as enhancers and promotors. The host cell may be of any type, including but not limited to mammalian, bacteria and yeast cells. Suitable bacterial cells include E. coli cells. Suitable mammalian cells include but are not limited to human embryonic kidney (HEK) 293T cells, HeLa cells, NIH 3T3 cells Chinese hamster ovary (CHO) cells and Cos cells.

This invention further provides a method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase which comprises: a) contacting a yeast cell cotransformed with a first plasmid which expresses in the cell a fusion protein comprising the wild-type or mutant p66 subunit polypeptide of HIV-1 reverse transcriptase and a second plasmid which expresses in the cell a fusion protein comprising the p51 subunit polypeptide of HIV-1 reverse transcriptase with the compound wherein the cell further comprises a reporter gene which is activated in the presence of a complex between the wild-type or mutant p66 subunit polypeptide and the p51 subunit polypeptide; b) determining the level of activity of the reporter gene in the cell in the presence of the compound; and c) comparing the level of activity of the reporter gene determined in step (b) with the level of activity of the reporter gene in the absence of the compound, wherein an increased level of activity of the reporter gene indicates that the compound is an activator or enhancer of the formation of the complex between the p51 subunit polypeptide of HIV-1 reverse transcriptase and the wild-type or mutant p66 subunit polypeptide of HIV-1 reverse transcriptase.

In an embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises full length bacterial protein LexA fused to the wild-type or mutant p66 subunit polypeptide of HIV-1 reverse transcriptase at amino acid position 1 of the N-terminal amino acid sequence of the full length bacterial protein LexA and the fusion protein expressed by the second plasmid further comprises amino acids at positions 768-881 of the C-terminal amino acid sequence of Gal4AD, wherein the C-terminal amino acid sequence of Gal4AD is fused at amino acid position 881 to one end of an influenza hemagglutinin (HA) epitope tag, wherein the p51 subunit polypeptide is fused to the second end of the influenza HA epitope tag.

In another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises full length bacterial protein LexA fused to the wild-type or mutant p66 subunit polypeptide of HIV-1 reverse transcriptase at amino acid position 1 of the N-terminal amino acid sequence of the full length bacterial protein LexA and the fusion protein expressed by the second plasmid further comprises amino acids at positions 768-881 of the C-terminal amino acid sequence of Gal4AD fused at position 881 to the p51 subunit polypeptide of HIV-1 reverse transcriptase.

In a further embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids 1-87 of the LexA DNA binding domain fused to the wild-type or mutant p66 subunit polypeptide of HIV-1 reverse transcriptase at amino acid position 87 and the fusion protein expressed by the second plasmid further comprises amino acids at positions 768-881 of the C-terminal amino acid sequence of Gal4AD, wherein the C-terminal amino acid sequence of Gal4AD is fused at amino acid position 881 to one end of an influenza hemagglutinin (HA) epitope tag, wherein the p51 subunit polypeptide is fused to the second end of the influenza HA epitope tag.

In another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids 1-87 of the LexA DNA binding domain fused to the wild-type or mutant p66 subunit polypeptide of HIV-1 reverse transcriptase at amino acid position 87 and the fusion protein expressed by the second plasmid further comprises amino acids at positions 768-881 of the C-terminal amino acid sequence of Gal4AD fused at position 881 to the p51 subunit polypeptide of HIV-1 reverse transcriptase.

In a still further embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises full length bacterial protein LexA protein fused at amino acid position 202 to a first end of a six alanine linker, wherein the wild-type or mutant p66 subunit polypeptide is fused at amino acid 1 to the second end of the six alanine linker and the fusion protein expressed by the second plasmid expresses further comprises amino acids at positions 768-881 of the C-terminal amino acid sequence of Gal4AD fused at position 881 to the p51 subunit polypeptide of HIV-1 reverse transcriptase.

In another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises full length bacterial protein LexA protein fused at amino acid position 202 to one end of a six alanine linker and the wild-type or mutant p66 subunit polypeptide fused at amino acid 1 to the other end of the six alanine linker and the fusion protein expressed by the second plasmid further comprises amino acids at positions 768-881 of the C-terminal amino acid sequence of Gal4AD wherein said C-terminal sequence is fused at amino acid position 881 to one end of an influenza hemagglutinin (HA) epitope tag and the p51 subunit polypeptide is fused at the other end of the HA epitope tag.

In a further embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD fused to a first end of an influenza hemagglutinin (HA) epitope and a six alanine linker fused at a first end to the second end of the influenza hemagglutinin (HA) epitope, wherein the wild-type or mutant p66 subunit polypeptide of HIV-1 reverse transcriptase is fused at amino acid 1 to the second end of the six alanine linker and the fusion protein expressed by second plasmid further comprises full length LexA bacterial protein LexA fused at amino acid 1 to the p51 subunit polypeptide of HIV-1 reverse transcriptase.

In another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD fused to a first end of an influenza hemagglutinin (HA) epitope and a six alanine linker fused at a first end to the second end of the influenza hemagglutinin (HA) epitope, wherein the wild-type or mutant p66 subunit polypeptide of HIV-1 reverse transcriptase is fused at amino acid 1 to the second end of the six alanine linker and the fusion protein expressed by the second plasmid further comprises the LexA DNA binding domain fused to amino acid position 1 of the p51 subunit polypeptide of HIV-1 reverse transcriptase.

In still another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD fused to a first end of an influenza hemagglutinin (HA) epitope and a six alanine linker fused at a first end to the second end of the influenza hemagglutinin (HA) epitope, wherein the wild-type or mutant p66 subunit polypeptide of HIV-1 reverse transcriptase is fused at amino acid 1 to the second end of the six alanine linker and the fusion protein expressed by the second plasmid further comprises the GAL4 DNA binding domain fused to amino acid position 1 of the p51 subunit polypeptide of HIV-1 reverse transcriptase.

In a further embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD, wherein the C-terminal amino acid sequence of Gal4AD is fused at amino acid position 881 to the wild-type or mutant p66 subunit polypeptide of HIV-1 reverse transcriptase, and the fusion protein expressed by second plasmid further comprises full length LexA bacterial protein LexA fused at amino acid position 1 to the p51 subunit polypeptide of HIV-1 reverse transcriptase.

In another embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD fused at amino acid position 881 to the wild-type or mutant p66 subunit polypeptide of HIV-1 reverse transcriptase and the fusion protein expressed by the second plasmid further comprises the LexA DNA binding domain fused to amino acid position 1 of the p51 subunit polypeptide of HIV-1 reverse transcriptase.

In a further embodiment of the above-described method of testing a compound to determine whether it is an activator or enhancer of formation of a complex between a wild-type or mutant p66 subunit polypeptide of HIV-1 reverse transcriptase and a p51 subunit polypeptide of HIV-1 reverse transcriptase, the fusion protein expressed by the first plasmid further comprises amino acids at positions 768-881 of the C-terminal amino acid sequence of Gal4AD fused at amino acid position 881 to the wild-type or mutant p66 subunit polypeptide of HIV-1 reverse transcriptase and the fusion protein expressed by the second plasmid further comprises the GAL4 DNA binding domain fused to amino acid position 1 of the p51 subunit polypeptide of HIV-1 reverse transcriptase.

The activators or enhancers determined by the above-described methods are useful for the preparation of drugs, as pharmaceutical compositions, which will enhance complex formation prematurely or inappropriately between the wild-type or mutant p66 subunit polypeptide of HIV-1 reverse transcriptase and the wild-type p51 subunit polypeptide, or p51 subunit polypeptide having the corresponding mutation as the mutant p66 subunit, of HIV-1 reverse transcriptase so as to kill the HIV-1 virus or render the HIV-1 virus inactive or incapable of infecting cells of a subject, i.e. lack the functions of an infected HIV-1 virus, including human subjects.

This invention also provides a method of making a pharmaceutical composition comprising an activator or enhancer of the formation of the complex between the wild-type or mutant p66 subunit polypeptide of HIV-1 reverse transcriptase and the p51 subunit polypeptide of HIV-1 reverse transcriptase which comprises: a) determining whether a compound is an activator or enhancer of the formation of the complex between the wild-type or mutant p66 subunit polypeptide of HIV-1 reverse transcriptase and the p51 subunit polypeptide of HIV-1 reverse transcriptase according to a method which comprises: i) contacting a yeast cell cotransformed with a first plasmid which expresses in the cell a fusion protein comprising the wild-type or mutant p66 subunit polypeptide of HIV-1 reverse transcriptase and a second plasmid which expresses in the cell a fusion protein comprising the p51 subunit polypeptide of HIV-1 reverse transcriptase with the compound wherein the cell further comprises a reporter gene which is activated in the presence of a complex between the wild-type or mutant p66 subunit polypeptide and the p51 subunit polypeptide; ii) determining the level of activity of the reporter gene in the cell in the presence of the compound; and iii) comparing the level of activity of the reporter gene determined in step (ii) with the level of activity of the reporter gene in the absence of the compound, wherein an increased level of activity of the reporter gene indicates that the compound is an activator or enhancer of the formation of the complex between the p51 subunit polypeptide of HIV-1 reverse transcriptase and the wild-type or mutant p66 subunit polypeptide of HIV-1 reverse transcriptase; and b) admixing the compound determined to be the activator in step (a)(iii) with a pharmaceutically acceptable carrier. Any of the above-described methods to determine whether a compound is an activator or enhancer of the formation of the complex between the wild-type or mutant p66 subunit polypeptide of HIV-1 reverse transcriptase and the p51 subunit polypeptide of HIV-1 reverse transcriptase may be used in the method of making a pharmaceutical composition comprising the determined activator or enhancer compound, but is not limited thereto, since one of skill will readily be able to substitute well known reporter genes for the reporter genes used in the examples herein. Moreover, one of skill is not limited to using the yeast cells exemplified in any of the above-described methods herein, but may modify the methods to use other eukaryotic cells, mammalian cells or cell lines such as 298 T cells.

Methods of treating a subject infected with HIV-1 include administering one or more of the herein-described compounds that enhance dimerization of the subunits of HIV-1 reverse transcriptase in a pharmaceutical compositions comprising: an activator or enhancer of the formation of the complex between the wild-type or mutant p66 subunit polypeptide of HIV-1 reverse transcriptase and the p51 subunit polypeptide of HIV-1 reverse transcriptase. One of skill will recognize that other pharmaceutical compositions may be administered to a subject infected with HIV-1 in conjunction with the pharmaceutical compositions provided by the methods set forth herein.

The invention also provides a pharmaceutical composition comprising an effective amount of any of the above-described activators or enhancers and a pharmaceutically acceptable carrier. In the subject invention an “effective amount” is any amount of an activator or enhancer which, when administered to a subject suffering from a disease or abnormality against which the activators or enhancers are effective, causes reduction, remission, or regression of the disease or abnormality. In the practice of this invention the “pharmaceutically acceptable carrier” is any physiological carrier known to those of ordinary skill in the art useful in formulating pharmaceutical compositions.

In one preferred embodiment the pharmaceutical carrier may be a liquid and the pharmaceutical composition would be in the form of a solution. In another equally preferred embodiment, the pharmaceutically acceptable carrier is a solid and the composition is in the form of a powder or tablet. In a further embodiment, the pharmaceutical carrier is a gel and the composition is in the form of a suppository or cream. In a further embodiment the compound or composition may be formulated as a part of a pharmaceutically acceptable transdermal patch.

A solid carrier can include one or more substances which may also act as flavoring agents, lubricants, solubilizers, suspending agents, fillers, glidants, compression aids, binders or tablet-disintegrating agents; it can also be an encapsulating material. In powders, the carrier is a finely divided solid which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Liquid carriers are used in preparing solutions, suspensions, emulsions, syrups, elixirs and pressurized compositions. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators. Suitable examples of liquid carriers for oral and parenteral administration include water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are useful in sterile liquid form compositions for parenteral administration. The liquid carrier for pressurized compositions can be halogenated hydrocarbon or other pharmaceutically acceptable propellant.

Liquid pharmaceutical compositions which are sterile solutions or suspensions can be utilized by for example, intramuscular, intrathecal, epidural, intraperitoneal or subcutaneous injection. Sterile solutions can also be administered intravenously. The compounds may be prepared as a sterile solid composition which may be dissolved or suspended at the time of administration using sterile water, saline, or other appropriate sterile injectable medium. Carriers are intended to include necessary and inert binders, suspending agents, lubricants, flavorants, sweeteners, preservatives, dyes, and coatings.

The activator(s) or compounds that enhance dimerization formation determined by the methods described above can be administered orally in the form of a sterile solution or suspension containing other solutes or suspending agents, for example, enough saline or glucose to make the solution isotonic, bile salts, acacia, gelatin, sorbitan monoleate, polysorbate 80 (oleate esters of sorbitol and its anhydrides copolymerized with ethylene oxide) and the like.

The activator(s) or compounds that enhance dimerization can also be administered orally either in liquid or solid composition form. Compositions suitable for oral administration include solid forms, such as pills, capsules, granules, tablets, and powders, and liquid forms, such as solutions, syrups, elixirs, and suspensions. Forms useful for parenteral administration include sterile solutions, emulsions, and suspensions.

Optimal dosages to be administered may be determined by those skilled in the art, and will vary with the particular inhibitor(s) or activator(s) in use, the strength of the preparation, the mode of administration, and the advancement of the disease condition or abnormality. Additional factors depending on the particular subject being treated will result in a need to adjust dosages, including subject age, weight, gender, diet, and time of administration.

This invention will be better understood from the Experimental Details that follow. However, one skilled in the art will readily appreciate that the specific methods and results discussed are merely illustrative of the invention as described more fully in the claims that follow thereafter.

Throughout this application, various publications are referenced within parentheses. Disclosures of these publications in their entireties are hereby incorporated by reference into this application to more fully describe the state of the art to which this invention pertains.

Experimental Details

First Series of Experiments

Materials and Methods

Bacterial and Yeast Strains

Saccharomyces cerevisiae strain CTY10-5d (MATa ade2 trp1-901 leu2-3, 112 his3-200 gal4-gal80-URA3::lexA-lacZ) contains an integrated GAL1-lacZ gene with the lexA operator (a gift from Stanley Fields, State University of New York, Stony Brook). The yeast strain HF7c contains CYC1-lacZ gene with three copies of the GAL4 responsive UASG 17-mer operator (CLONTECH). Escherichia coli mutator strain XL1-Red (Stratagene) was used for random mutagenesis whereas XL1-Blue (Stratagene) was used to amplify the mutated library. KC8 (CLONTECH), an auxotrophic leuB, trpc and hisB E. coli strain, was used to isolate plasmids from yeast. E. coli strains M15 and BL21 were used to express p66-His and glutathione S-transferase-tagged p51 (GST-p51) respectively (see below).

Yeast Methods

Transformation of yeast and the qualitative β-galactosidase (β-gal) colony lift assay were as published (19). Quantification of protein-protein interactions was determined using the β-gal liquid assay performed on permeabilized yeast grown from three independent transformants using orthonitrophenyl-β-D-galactopyranoside as substrate (19).

Protein Expression and RT Activity

Fusion protein expression in yeast was determined by Western blot of lysates with Gal4AD polyclonal antibodies (Upstate Biotechnology, Lake Placid, N.Y.), anti-lexA polyclonal antibodies (Invitrogen) and HIV-1 RT polyclonal (Intracel, Cambridge, Mass.) or 5B2 monoclonal antibody (20). Immunodetection was with ECL-Plus (Amersham). To measure RT activity, yeast lysates were prepared by glass bead disruption (19) and enzyme activity was determined in exogenous assays (21) and quantified by phosphoimager analysis.

Yeast Shuttle Vectors

pSH2-1 (22) and pLex202-PL (23) express the lexA DNA binding domain (lexA₈₇) and the full-length lexa protein (leXA₂₀₂), respectively. pGBT9 and pAS2-1, both containing the GAL4 DNA binding domain (GAL4 BD), were purchased from CLONTECH. pNLexA allows expression of proteins fused to the N terminus of full-length lexA₂₀₂ (OriGene Technologies, Rockville, Md.). PGADNOT (18) and PACTII (24) allow expression of proteins fused to the Gal4 activation domain (GAL4 AD). pACTII also contains the influenza hemagglutinin (HA) epitope tag located between GAL4AD and the polylinker.

Construction of HIV-1 RT Fusions in Yeast Vectors

Constructs and expressed fusion proteins are as described in FIG. 1. The RT sequence for constructing the following expression vectors was amplified from HIV-1 molecular clone pNLenv-1 (containing the HIVNL43 sequence) (25). The p66 amplimers were cloned into the BamHI-SalI sites of pGBT9, pSH2-1, pLex202-PL, PACTII and PGADNOT; the BamH1-XhoI sites of PACTII; and the EcoRI-BamHI sites of pNLexA. p51 amplimers were cloned into the BamHI-SalI sites of these vectors except for cloning into PACTII, where the BamH1-XhoI sites were used. The HXB2 RT sequence from pHXB2gpt (26) was used to construct p66HXAlaLex202 and p51HXGADNOT.

Construction of HIV-1 RT Deletion Mutants

All p66 deletion mutants were prepared by cloning PCR amplimers into the BamHI-SalI sites of pSH2-1. Fingers, palm, connection, thumb and RNase H domains of HIV-1 RT are denoted F, P, C, T and R respectively. pT+C+RSH2-1 (encoding lexA₈₇-T+C+R) contains RT (from HIVNL43) codons 236-560. pC+RSH2-1 (encoding lexA₈₇-C+R) contains codons 322-560 while pRSH2-1 (encoding lexA₈₇-R) comprises codons 425-560. All p51 deletion mutants were prepared by cloning of PCR amplimers into the BamHI-XhoI sites of PACTII. pF+P+T-ACTII (encoding Gal4AD-HA-F+P+T) includes RT codons 1-325 and pF+P-ACTII (encoding Gal4AD-HA-F+P) has codons 1-244. p51Δ13ACTII (encoding Gal4AD-HA-51Δ13) contains RT codons 1-426. p51Δ26GADNOT (encoding Gal4AD-51Δ26) was obtained by random mutagenesis of p51GADNOT in XL1-Red.

Construction of RT Fusions with the L234A Mutation and Random Mutagenesis of p66Ala234Lex202 and Selection of Revertants

p66Ala234Lex202 (encoding lexA₂₀₂-Ala-66L234A) was made by inserting p66 from p6HprotL234A (a gift from Vinayaka Prasad, Albert Einstein College of Medicine, Bronx N.Y.) into the BamHI/SalI sites of pLex202-PL. p51234GADNOT (encoding Gal4AD-51L234A) was made by insertion of p51 from p6HprotL234A into the BamHI-SalI sites of PGADNOT. Second-site mutations restoring dimerization to lexA₂₀₂-Ala-66L234A were generated by propagation of p66Ala234Lex202 in XL1-Red (Stratagene). Two independent pools were prepared. CTY10-5d was cotransformed with the mutagenized library and either p51234GADNOT or p51GADNOT. Blue colonies were picked from β-gal colony lift assays and clonally purified. p66 DNA from isolated plasmids were recloned into a nonmutated pLex202-PL backbone and reintroduced into CTY10-5d to confirm the phenotype. Mutations present in p66 were determined by automated nucleotide sequencing.

Site Directed Mutagenesis

p66 with genotype D110G was prepared from a p66 clone containing both D110G and L234A obtained by random mutagenesis by backmutation of codon 234 to wild-type. p66 with either the W402R or W406R substitutions were prepared by subcloning a Bsp1286I-SalI fragment (600 bp) from the clones obtained by random mutagenesis of L234A with wild-type BamHI-Bsp1286I fragment (1,080 bp) from p66HXAlaLex202 into pLex202-PL.

In vitro Heterodimerization

Plasmids expressing wild-type and p66 mutants with a histidine tag at the C-terminus (p66-His) were constructed by cloning the p66 coding region into the SphI -BglII site of pQE-70 (Qiagen, Chatsworth, Calif.). The C-terminal tag was appended as described previously [clone 3 (27)]. Glutathione S-transferase tagged p51 (GST-p51) was prepared by subcloning the BamHI-SalI fragment from p51HXGADNOT into pGEX5X-3 (Amersham Pharmacia). Cells were induced and then lysed by the addition of 1 mg/ml of lysozyme to 1 ml of lysis buffer [50 mM sodium phosphate buffer (pH 7.8), 500 mM NaCl, 0.5% Nonidet P-40, 5 mM DTT, and 1 μg/ml each of pepstatin A, aprotinin and leupeptin] and clarified. Lysates were combined and incubated for 16 hrs at 4° C. The heterodimer was captured on Glutathione Sepharose 4B beads and unbound subunits were removed by washing with lysis buffer. Heterodimer bound to beads were resolved by SDS/PAGE. For quantification of RT activity, dimers were eluted from beads with 10 mM reduced glutathione in 50 mM Tris (pH 8.0). Samples were assayed for DNA polymerase activity on homopolymeric template-primers for various times, and the activity was determined from the initial slope of the linear phase of the time course. Western blot confirmed equal recovery of GST-51 protein in each sample.

Results

Expression of RT Fusion Proteins and RT Activity

The stable expression of p66 was tested in several contexts, as either Gal4BD or LexA fusions, and using a six alanine linker to separate p66 from its fusion partner. p66 fused to the C terminus of lexA₈₇, the C or N termini of lexA₂₀₂ (with or without a six alanine spacer), and in a variety of contexts to the Gal4AD were all stably expressed (FIG. 1). In contrast, p66 fused to the C terminus of the Gal4BD (Gal4BD-66) was not expressed in yeast at detectable levels (FIG. 1). The smaller RT subunit, p51, was well expressed as fusions with the Gal4BD, Gal4AD, and both leXA₈₇ and lexA₂₀₂.

We examined whether the bait fusions encoded by p66SH2-1, p66AlaLex202 and p66NLexA exhibited RT activity in yeast. All three fusion proteins demonstrated high levels of RT activity compared with protein lysates from yeast transformed with an empty vector (data not shown). These data suggest that the p66 fusion proteins are functional and in a conformation consistent with measurable catalytic activity.

Heteromeric Interactions of p66 and p51 by Transactivation in the Two-Hybrid System

To test whether the Y2H system could detect the interaction of the p66/p51 heterodimer, we cotransformed yeast reporter strains with plasmids expressing p66DNA BD and p51DNA AD fusion proteins (Table 1). β-gal activity expressed in yeast, which indicates the strength of the interaction between the fusion proteins, was assessed by both qualitative and quantitative assays. The p66 bait fusions expressed from p66SH2-1, p66AlaLex202 and p66NLexA interacted with Gal4AD-p51 domain fusions (Table 1) but not with Gal4AD alone (Table 1). The strongest interactions were observed with p66 baits lexA₂₀₂-Ala-66 and 66-lexA₂₀₂. Moreover, p51 expressed in PACTII gave a stronger signal than p51GADNOT when coexpressed with p66 fusion baits. Despite the stable expression of the p66 fusion protein, lexA₂₀₂-66, no significant interaction with p51 was detected (FIG. 1). Moreover, lexA₂₀₂-66 yielded the same weak signal with the empty Gal4AD vector, PGADNOT, indicating that this version of p66 is weakly self-activating even without a partner.

TABLE 1
Interaction of p66 binding domain fusions with p51
activation domain fusions in the Y2H system

| | | β-gal activity | |
|---|---|---|---|
| Constructs | Operator | Colony* | Liquid^{†} |
| p66SH2-1 + pGADNOT | lexA | − | ND |
| p66SH2-1 + pACTII | lexA | − | 0.02 |
| p66SH2-1 + p51GADNOT | lexA | ++ | 0.5 |
| p66SH2-1 + p51ACTII | lexA | +++ | 3.5 |
| p66AlaLex202 + pGADNOT | lexA | − | ND |
| p66AlaLex202 + pACTII | lexA | − | 0.04 |
| p66AlaLex202 + p51GADNOT | lexA | +++ | 1.6 |
| p66AlaLex202 + p51ACTII | lexA | +++ | 7.7 |
| p66NLexA + pGADNOT | lexA | − | 0.06 |
| p66NLexA + pACTII | lexA | − | 0.04 |
| p66NLexA + p51GADNOT | lexA | +++ | 6.6 |
| p66NLexA + p51ACTII | lexA | +++ | 25.0 |
| p66Lex202 + pGADNOT | lexA | +/− | ND |
| p66Lex202 + p51GADNOT | lexA | +/− | ND |
| p66GBT9 + pGADNOT^{‡} | UAS_{G} | − | ND |
| p66GBT9 + p51GADNOT^{‡} | UAS_{G} | − | ND |

Yeast strain CTY10-5d or ^{‡}HF7c were transformed with plasmids encoding p66 bait and p51 prey fusions. Fusion proteins encoded by plasmids are described in Materials and Methods and FIG. 1.
*Transformants were lifted onto nitrocellulose and subjected to the β-gal colony lift assay to determine intensities of blue color produced; +++, strong blue in 1 h; ++, blue in 1 h; +/−, weak blue in 3 h; −, white; ND, not done.

We also showed that heteromeric interactions between p66 and p51 could be detected in the reciprocal configuration with p51 as either a LexA or Gal4BD fusion and p66 as a Gal4AD fusion (Table 2). The demonstration of heteromeric dimerization of p66 and p51 in different contexts strongly suggests that the interaction is specific. Tests for interaction with five unrelated proteins showed no signal (data not shown), providing further evidence for the specificity of RT heterodimerization in yeast.

TABLE 2
Interaction of p51 binding domain fusions with p66
activation domain fusions in the Y2H system

β-gal activity

| Constructs | Operator | Colony* | Liquid^{†} |
|---|---|---|---|
| p51SH2-1 + pGADNOT | lexA | − | 0.06 |
| p51SH2-1 + pACTII | lexA | − | 0.05 |
| p51SH2-1 + p66AlaACTII | lexA | ++ | 1.2 |
| p51Lex202 + pACTII | lexA | − | 0.05 |
| p51Lex202 + p66AlaACTII | lexA | +++ | 3.2 |
| p51AS2-1 + pACTII^{‡} | UAS_{G} | − | ND |
| p51AS2-1 + p66AlaACTII^{‡} | UAS_{G} | ++ | ND |

Yeast strain CTY10-5d or ^{‡}HF7c were transformed with plasmids encoding p51 bait and p66 prey fusions. Fusion proteins encoded by plasmids are described in Materials and Methods and FIG. 1.
*As defined in Table 1.
^{‡}As defined in Table 1.

Homomeric Interactions

The interaction of the RT heterodimer p66/p51 has a dissociation constant of 10⁻⁹ M, whereas the dissociation constants for the p66 and p51 homodimers are 10⁻⁶ M and 10⁻⁵ M, respectively (9). We were unable to detect p51 homodimerization when CTY10-5d was cotransformed with either p51SH2-1 or p51Lex202 baits and p51ACTII prey (data not shown). In contrast, p66 homodimerization could be detected when yeast was cotransformed with p66NlexA bait and p66AlaACTII prey (β-gal activity 0.3 Miller units). p66 homodimerization of these two constructs was 100-fold weaker compared to the interaction of p66NlexA with p51ACTII (Table 1). The strength of the interactions observed in vivo are consistent with biochemical data.

p66 Domains that Interact with p51

We used the Y2H RT dimerization assay to map the regions of p66 required for binding to p51 (FIG. 2). A series of mutants with sequential deletions in the polymerase subdomains were prepared as C-terminal fusions with lexA₈₇. Deletion of the fingers and palm subdomains (lexA₈₇-T+C+R) did not significantly affect binding to Gal4AD-HA-51. A further deletion of the thumb subdomain (lexA87-C+R) resulted in reduced β-gal activity (FIG. 2). Expression of the RNase H domain alone was not sufficient for interaction with p51. This lack of interaction was not attributable to an aberrant RNase H conformation, as lexA₈₇-R also interacted as strongly as leXA₈₇-66 with a cellular protein, diaphorase, that we find interacts with the RNase H domain of RT in the Y2H system (results not shown). None of the bait fusions demonstrated activation of the lacZ reporter gene when coexpressed with Gal4AD-HA alone, excluding the possibility of nonspecific self-activation by the bait fusions (results not shown). These data suggest that the connection and RNase H subdomains of p66 are sufficient for interaction with p51.

The C Terminus of p51 is Required for Interaction with p66

It has previously been shown biochemically that deletion of as little as 25 amino acids from the C terminus of p51 can prevent dimerization to p66 (15). To ascertain whether this effect could be observed under physiological conditions in the Y2H system, we constructed a series of C-terminal deletion mutants of p51 prey fusions and assayed interaction with p66 bait. Deletion of 13 amino acids from the C terminus of p51 had little effect (1.8-fold decrease) on dimerization with p66 (FIG. 3). However, deletions of 26 amino acids and greater abrogated RT dimerization, indicating the importance of the C-terminal 26 amino acids of p51 in these interactions. These results also suggest that the system faithfully recapitulates the behavior of the enzyme as studied in vitro.

L234A in p66 Subunit Inhibits RT Dimerization

The Y2H RT dimerization assay would be most useful if it could be applied to the analysis of single amino acid substitutions that affect heteromeric interactions. To test the system, we introduced the L234A primer grip mutation, previously shown biochemically to inhibit p66/p51 association (10), into both RT subunits. The presence of L234A in both p66 and p51 totally inhibited RT dimerization as observed by a 53-fold decrease in the β-gal signal compared to wild-type proteins (FIG. 4). To assess the effect of L234A in individual subunits CTY10d-5 was cotransformed with constructs expressing either p66 mutant bait and wild-type p51 prey, or p66 wild-type bait with p51 mutant prey. Less than a two-fold decrease in the signal compared with wild-type fusions was observed when the L234A mutant p51 (Gal4AD-51L234A) was coexpressed with the wild-type fusion lexA₂₀₂-Ala-66 (FIG. 4). However, a 32-fold inhibition was observed for the interaction of the mutant lex₂₀₂-Ala-66L234A with wild-type Gal4AD-51. These data suggest that L234A affects dimerization predominantly through p66, as has been previously reported (10). Analysis of fusion protein expression in yeast by Western blot analysis revealed that all fusion proteins, including the L234A mutants, were stably expressed (results not shown).

Second-Site Mutations that Restore Heterodimerization and RT Activity to the p66L234A Mutant

To gain insight into the mechanism of inhibition of RT dimerization by L234A, we attempted to select for second-site suppressor mutations in p66 that restore dimerization with p51. To select for p66 mutants with restored dimerization, CTY10-5d was cotransformed with a library generated by mutagenesis of p66AlaL234ALex202 and a plasmid expressing either Gal4AD-51 or the Gal4AD-51-L234A mutant. A total of 25,000 colonies from each of two independently mutated libraries were screened. Six and five blue colonies were obtained when lex₂₀₂-Ala-66L234A was cotransformed with Gal4AD-51 and Gal4AD-51-L234A, respectively. CTY10-5d was retransformed with each isolated library plasmid and with either p51HXGADNOT or p51L234AGADNOT; the recovered clones showed restored binding activity with both p51 fusion proteins. Five types of mutations were observed (Table 3). Single amino acid changes in the clones that retained the L234A change included D110G, D186V, W402R and W406R. The remaining three clones had reverted to wild-type at codon 234 (Table 3).

TABLE 3
Second site mutations in lexA₂₀₂66HXL234A
that restore dimerization to p51

β-gal activity

| Constructs | No. of Clones | Colony* | Liquid^{†} |
|---|---|---|---|
| Wild Type | NA | +++ | 3.2 |
| L234A | NA | − | 0.1 |
| D110G | NA | +++ | 6.9 |
| W402R | NA | +++ | 7.1 |
| W406R | NA | +++ | 5.8 |
| L234A; D110G | 3 | +++ | 1.5 |
| L234A; D186V | 1 | +/− | 0.2 |
| L234A; W402R | 3 | +++ | 7.1 |
| L234A; W406R | 1 | +++ | 6.1 |
| L234 | 3 | +++ | 4.7 |

Yeast strain CTY10-5d was cotransformed with p51HXGADNOT and various clones expressing lexA₂₀₂-Ala-66HX fusions with mutations in p66 as indicated. NA, not applicable.
*As defined in Table 1.
^{‡}As defined in Table 1.

Two of the changes are at the catalytically essential aspartyl residues D110 and D186. These residues are not located at the dimer interface, and mutations at these residues result in an inactive RT (28)(FIG. 5). A variant p66 containing D110G alone, without L234A, gave a 2-fold stronger β-gal signal than wild-type p66 for heterodimerization and was 4.6-fold stronger compared with clones containing both L234A and D110G. Partial restoration of dimerization by D110G suggests that conformational changes at the active site compensate for structural changes mediated by L234A.

The second set of mutations, W402R and W406R, are located at the dimer interface (FIG. 5) in a tryptophan repeat region which is highly conserved among HIV-1, HIV-2 and closely related simian immunodeficiency virus RTs (29). In the L234A genetic background, these mutations resulted in a dramatic increase in the β-gal signal over the parent and yielded a 2-fold higher signal for heterodimerization compared with wild-type RT fusions (Table 3). W402R and W406R in a wild-type genetic background had the same enhanced β-gal activity as the restored mutants (Table 3). Therefore, the mutations in the tryptophan repeat motif may enhance the interaction with GAL4AD-51 independently of the L234A mediated defect.

To confirm that the second-site mutations could restore heterodimerization to the L234A parent in an alternative assay, we examined the binding of these p66 mutants to p51 in vitro. Bacterial lysates containing GST-p51 or wild-type and mutant p66-His were incubated together, and heterodimers were captured on Glutathione Sepharose 4B beads. As expected, wild-type p66 dimerized with GST-p51 whereas the p66L234A mutant did not (FIG. 6A). Restoration of dimerization by D110G, W402R or W406R in the L234A parent was observed (FIG. 6A), thus confirming our observations in the Y2H assay.

To determine whether restoration of heterodimerization was associated with enhanced DNA polymerase activity, heterodimers eluted from beads were assayed for RT activity (FIG. 6C). GST-p51 had significant background activity compared with wild-type enzyme. The enzyme resulting from incubation with p66L234A had the same background activity. As expected, heterodimers comprising p66L234A containing the active site mutation D110G also had only background activity. Interestingly, both W402R and W406R mutations not only restored heterodimerization to the L234A parent but also increased RT activity, even above levels of the wild-type control (FIG. 6C).

Discussion

In this study we have shown that fusions of p66 and p51 can be stably expressed in yeast and can heterodimerize in reciprocal configurations. The presence of spacers in the form of alanine or an HA tag may have been an important aspect for stronger interactions in the Y2H assay. Moreover, we have validated the Y2H assay by comparing previously described effects of p51 deletions and the L234A substitution on heterodimerization. We have also shown how this assay can further the study of the HIV-1 RT structure-function by the identification of second-site mutations that restore RT dimerization.

The palm, connection, and RNase H domains of p66 make major contacts with p51. An indication that the palm region is important is the destabilization of the p66/p51 heterodimer by the nonnucleoside RT inhibitor 2′,5′-bis-O-(tert-butylidimethylsilyl) -3′-spiro-5″-4″-amino-1″,2″-oxothiole-2″,2″-dioxide)]-b-D- pentofuranosyl (TSAO) by its interaction between the palm subdomain of p66 and the β7-β8 loop in the fingers subdomain of p51 (30, 31). Preliminary tests of the addition of TSAO to our in vitro binding assays confirm the ability of the drug to reduce heterodimerization (data not shown). Tests of the related drug TSAOe³T showed a more modest destabilization only detectable in the presence of denaturants (31). Deletion mapping of the p66 domains required for interaction with p51 suggests that the presence of the connection and RNase H domains are sufficient for interaction with p51 in the Y2H system. It is surprising that the deletion of the palm domain had little effect on binding to p51 as this p66 subdomain provides a major contact with p51 (9); however, the connection and RNase H domains may provide a sufficient surface for saturating the signal in yeast.

Truncation of the C terminus of p51 revealed that a 13-amino acid deletion had little effect on dimerization with p66, but a deletion of 26 amino acids abrogated heterodimerization as seen in the Y2H assay. These data are consistent with previous in vitro studies (15). All C-terminal truncation mutants were stably expressed in yeast, excluding the possibility of decreased expression affecting the signal. It is possible that these C-terminal residues may have a direct role in dimerization; or the deletion of these residues may effect the structural integrity or correct positioning of the structural elements a-L and P-20 (5,15). These elements contain the tryptophan repeat motif, which has been proposed to play an important role in HIV-1 dimerization (29, 32).

It has been shown that the L234A substitution inhibits RT dimerization in yeast most dramatically when present on the p66 subunit of HIV-1 RT, as previously seen in vitro (10). L234A is located in the primer grip region of p66 (5) and is highly conserved among avian, primate and murine RTs (33). To help determine the mechanism by which L234A affects heterodimerization, we selected for second-site mutations restoring p66/p51 association. Aside from clones which had reverted to the wild-type L234, we observed two classes of mutants: those with alterations either in the tryptophan repeat or in the polymerase active site (FIG. 5). Both classes of suppressors were also shown to restore binding of the mutant p66 subunit to p51 as measured in an in vitro binding assay (FIG. 6A). L234A is not at the dimer interface, and it has been proposed that it affects dimerization by indirectly affecting contacts between P95 in the palm of p66 with residues in the β7-β8 loop of p51 (11). The mutations W402R and W406R are distant from this region, being located in the connection subdomain which contacts the p51 connection domain in the heterodimer. The appearance of a basic residue in both codon 402 and 406 suggests a charge interaction with an acidic residue in p51 or alternatively an increase in electrostatic potential between the surfaces at the connection domain interface.

The recovery of second-site suppressor mutations at the catalytically essential aspartyl residues suggests that there is a relationship between dimerization and active site residues. Neither D186V nor D110G make obvious contacts with L234A, although both are in the same palm subdomain (FIG. 5) (2). Interaction between the NNRTI binding site, which includes L234, and the RT catalytic site has been suggested by both structural and enzymatic data explaining the mechanism of resistance to NNRTIs (34, 35). The D110G or D186V changes would probably result in loss of one of the two magnesium ions bound to the active site (36). A loss of chelated magnesium in addition to a glycine change at 110 may lead to increased flexibility in that region, thus affecting dimerization. Determination of the crystal structure of the D110G RT mutant will help resolve these issues.

Heterodimerization of HIV-1 has been suggested as a target for chemotherapeutic intervention (7). To date, there are no HIV-1 RT dimerization inhibitors being used in the clinic. Nevertheless, there are several reports of HIV-1 and HIV-2 RT dimerization inhibitors based on peptides representing the conserved tryptophan repeat region of RT (32, 37). These peptides have been shown to prevent the association of p66/p51 (32) and have demonstrable in vitro anti-HIV-1 activity (37). TSAO has been shown to destabilize the p66/p51 heterodimer and may represent a nonpeptide RT dimerization inhibitor (30). In preliminary tests of this drug for its effects on heterodimerization in the Y2H system, we saw no inhibition of β-gal activity (data not shown). However, the possibility that the drug is not taken up by yeast cannot be ruled out. The availability of a Y2H assay for RT dimerization will facilitate the screening for other such inhibitors of this process according to the methods set forth herein.

### REFERENCES FOR FIRST SERIES OF EXPERIMENTS

1. di Marzo Veronese, F., Copeland, T. D., DeVico, A. L., Rahman, R., Oroszlan, S., Gallo, R. C. & Sarngadharan, M. G. (1986) Science 231, 1289-1291.

2. Rodgers, D. W., Gamblin, S. J., Harris, B. A., Ray, S., Culp, J. S., Hellmig, B., Woolf, D. J., Debouck, C. & Harrison, S. C. (1995) Proc. Natl. Acad. Sci. USA 92, 1222-1226.

3. Ren, J., Esnouf, R., Garman, E., Somers, D., Ross, C., Kirby, I., Keeling, J., Darby, G., Jones, Y., Stuart, D., et al. (1995) Nat. Struct. Biol. 2, 293-302.

4. Huang, H., Chopra, R., Verdine, G. L. & Harrison, S. C. (1998) Science 2 82, 1669-1675.

5. Jacobo-Molina, A., Ding, J., Nanni, R. G., Clark, A. D., Jr., Lu, X., Tantillo, C., Williams, R. L., Kamer, G., Ferris, A. L., Clark, P., et al. (1993) Proc. Natl. Acad. Sci. USA 90, 6320-6324.

6. Kohlstaedt, L. A., Wang, J., Friedman, J. M., Rice, P. A. & Steitz, T. A. (1992) Science 256, 1783-1790.

7. Restle, T., Muller, B. & Goody, R. S. (1990) J. Biol.

Chem. 265, 8986-8988.

8. Becerra, S. P., Kumar, A., Lewis, M. S., Widen, S. G., Abbotts, J., Karawya, E. M., Hughes, S. H., Shiloach, J. & Wilson, S. H. (1991) Biochemistry 30, 11707-11719.

9. Wang, J., Smerdon, S. J., Jager, J., Kohlstaedt, L.

A., Rice, P. A., Friedman, J. M. & Steitz, T. A. (1994) Proc. Natl. Acad. Sci. USA 91, 7242-7246.

10. Ghosh, M., Jacques, P. S., Rodgers, D. W., Ottman, M., Darlix, J. L. & Le Grice, S. F. (1996) Biochemistry 35, 8553-8562.

11. Wohrl, B. M., Krebs, R., Thrall, S. H., Le Grice, S. F. J., Scheidig, A. J. & Goody, R. S. (1997) J. Biol. Chem. 272, 17581-17587.

12. Goel, R., Beard, W. A., Kumar, A., Casas-Finet, J. R., Strub, M. P., Stahl, S. J., Lewis, M. S., Bebenek, K., Becerra, S. P., Kunkel, T. A., et al. (1993) Biochemistry 32, 13012-13018.

13. Divita, G., Restle, T. & Goody, R. S. (1993) FEBS Lett. 324, 153-158.

14. Debyser, Z. & De Clercq, E. (1996) Protein Sci. 5, 278-286.

15. Jacques, P. S., Wohrl, B. M., Howard, K. J. & Le Grice, S. F. (1994) J. Biol. Chem. 269, 1388-1393.

16. Fields, S. & Song, 0. (1989) Nature (London)340, 245-246.

17. Kalpana, G. V. & Goff, S. P. (1993) Proc. Natl. Acad. Sci. USA 90, 10593-10597.

18. Luban, J., Bossolt, K. L., Franke, E. K., Kalpana, G. V. & Goff, S. P. (1993) Cell 73, 1067-1078.

19. Rose, M. D., Winston, F. & Hieter, P. (1990) Methods in Yeast Genetics: A Laboratory Course Manual (Cold Spring Harbor Laboratory Press, New York),

20. Szilvay, A. M., Nornes, S., Haugan, I. R., Olsen, L., Prasad, V. R., Endresen, C., Goff, S. P. & Helland, D. E. (1992) J. Acquired Immune Defic. Syndr. 5, 647-657.

21. Telesnitsky, A., Blain, S. & Goff, S. P. (1995) Methods Enzymol. 262, 347-362.

22. Hanes, S. D. & Brent, R. (1989) Cell 57, 1275-1283.

23. Ruden, D. M., Ma, J., Li, Y., Wood, K. & Ptashne, M. (1991) Nature (London) 350, 250-252.

24. Legrain, P., Dokhelar, M. C. & Transy, C. (1994) Nucleic Acids Res. 22, 3241-3242.

25. Maldarelli, F., Martin, M. A. & Strebel, K. (1991) J. Virol. 65, 5732-5743.

26. Ratner, L., Haseltine, W., Patarca, R., Livak, K. J., Starcich, B., Josephs, S. F., Doran, E. R., Rafalski, J. A., Whitehorn, E. A., Baumeister, K., et al. (1985) Nature (London) 313, 277-284.

27. Maier, G., Dietrich, U., Panhans, B., Schroder, B., Rubsamen-Waigmann, H., Cellai, L., Hermann, T. & Heumann, H. (1999) Eur. J. Biochem. 261, 10-18.

28. Larder, B. A., Purifoy, D. J., Powell, K. L. & Darby, G. (1987) Nature (London) 327, 716-717.

29. Baillon, J. G., Nashed, N. T., Kumar, A., Wilson, S. H. & Jerina, D. M. (1991) New Biol. 3, 1015-1019.

30. Harris, D., Lee, R., Misra, H. S., Pandey, P. K. & Pandey, V. N. (1998) Biochemistry 37, 5903-5908.

31. Sluis-Cremer, N., Dmitrienko, G. I., Balzarini, J., Camarasa, M. J. & Parniak, M. A. (2000) Biochemistry 39, 1427-1433.

32. Divita, G., Restle, T., Goody, R. S., Chermann, J. C. & Baillon, J. G. (1994) J. Biol. Chem. 269, 13080-13083.

33. Georgiadis, M. M., Jessen, S. M., Ogata, C. M., Telesnitsky, A., Goff, S. P. & Hendrickson, W. A. (1995) Structure (London) 3, 879-892.

34. Esnouf, R., Ren, J., Ross, C., Jones, Y., Stammers, D. & Stuart, D. (1995) Nat. Struct. Biol. 2, 303-308.

35. Spence, R. A., Kati, W. M., Anderson, K. S. & Johnson, K. A. (1995) Science 267, 988-993.

36. Patel, P. H., Jacobo-Molina, A., Ding, J., Tantillo, C., Clark, A. D., Jr., Raag, R., Nanni, R. G., Hughes, S. H. & Arnold, E. (1995) Biochemistry 34, 5351-5363.

37. Morris, M. C., Robert-Hebmann, V., Chaloin, L., Mery, J., Heitz, F., Devaux, C., Goody, R. S. & Divita, G. (1999) J. Biol. Chem. 274, 24941-24946.

38. Kraulis, P. J. (1991) J. Appl. Crystallogr. 24, 946-950.

39. Merrit, E. A. & Bacon, D. J. (1997) Methods Enzymol. 277, 505-524.

40. Kabsch, W. & Sander, C. (1983) Biopolymers 22, 2577-2637.

Second Series of Experiments

Materials and Methods

Antiviral drugs

The drugs used in this study were: carboxanilides such as N-[4-chloro-3-(3-methyl-2-butenyloxy)phenyl]-2-methyl-3-furancarbothioamide (UC781), UC10, UC38, UC84 (Damian, F. et al J. Pharm. Pharmacol. 53:1109-1116, 2001; Ren, J. et al Biochemistry 37:14394-14403, 1998;24, 25), Uniroyal Chemical Ltd (Middlebury, Conn.); efavirenz (EFV) (21), DuPont Merck (Wilmington Del.); delavirdine (BHAP)(26), Amersham Pharmacia and Upjohn (Kalamazoo, Mich); nevirapine (19), Roxanne Laboratories (Redding Conn); (S)-4-isopropoxycarbonyl-6-methoxy-3 -(methylthiomethyl)-3,4-dihydroquinoxaline-2(1H)-thione (HBY 097) (27), Hoechst-Bayer (Frankfurt, Germany); (−)-(S)-8-chloro-4,5,6,7-tetrahydro-5-methyl-6-(3-methyl-2-butenyl)imidazo[4,5,1-jk] [1,4] benzodiazepin-2 (1H)-thione monohydrochloride (8-Cl-TIBO) (28) and a-APA (29), Janssen Research Foundation (Beerse, Belgium). All drugs were dissolved in dimethyl sulfoxide at a concentration of 10 mg/ml for use in Y2H and in vitro assays.

Yeast and Bacterial Strains and Yeast Methods

Yeast and bacterial strains were as described previously (22). Transformation of yeast, the qualitative P-gal colony lift assay and the quantitative P-gal liquid assay were as previously described (22).

Construction of HIV-1 RT Fusions in Yeast Expression Vectors

The construction of p66SH2-1, p51SH2-1, p66GADNOT, p51GADNOT and p51ACTII which express the wild type p66 and p51 fusion proteins lexA87-66, lexA87-51, Gal4AD-66, Gal4AD-51 and Gal4AD-HA-51, respectively were as described previously (22). p66L234ASH2-1 (encoding lexA87-66L234A) was made by cloning the PCR amplification product from the RT region of p66AlaL234ALex202 (22) into the BamHI-SalI sites of pSH2-1. p51234ACTII (encoding Gal4AD-HA-51L234A) was constructed by subcloning the p51 BamHI-SalI fragment from p51234GADNOT (22) into PACTII. p66W401ASH2-1 (encoding lexA87-66W401A), p5lW401AACTII (encoding Gal4AD-HA-51W401A) and p5lW401AGADNOT (encoding Gal4AD-51W401A) were made by PCR amplification of the RT region from plasmid pALRT-78S(A402) (a gift from John McCoy) and cloned into the BamHI-SalI sites of pSH2-1, pGADNOT or the BamHI-XhoI sites of pACTII. p66Y181CSH2-1 containing the Y181C mutation in p66 of the lexA87-66 fusion protein was prepared by site-directed mutagenesis using the Gene Editor Kit (Promega, Madison, Wis.) according to the manufacturer's protocol. Other mutations in p66, such as F227L, G190A, G190E, G190S, K101E, K103N, K238T, L1001, P225H, V106A, V106I, V108I, Y188H, Y188L, including one, two, three, four, five and more mutations may also be prepared using site-directed mutagenesis (Bachler, L. et al ibid).

Construction of HIV-1 RT Fusions in Bacterial Expression Vectors

Wild-type and p66 mutants (either L234A or W401A) were cloned into the SphI-BglII site of pQE-70 (Qiagen, Chatsworth, Calif.) (22). Glutathione S-transferase-tagged p51 (GST-p51) and mutants containing either the W401A or L234A substitutions were constructed by cloning the p5i encoding fragments into the BamHI-SalI site of pGEX5X-3 (Amersham Pharmacia Biotech) (22).

Y2H RT Heterodimerization Assays for Measuring Effect of NNRTIs on β-gal Activity.

CTY10-5d transformed constructs expressing p66 bait and p51 prey fusions were grown overnight to stationary phase in synthetic complete medium without histidine and leucine and containing 2% glucose (SC-His-Leu). 2.5 ml of media with or without drug were inoculated with 0.0125-0.25 OD600 units of CTY10-5d. Yeast were grown with aeration at 30° C. to OD600=0.5. The equivalent of 1 OD600 unit was pelleted for each treatment and subjected to a quantitative _-gal liquid assay.

Coimmunoprecipitation of p66 and p51 in Yeast Lysates.

Cultures (30 ml) containing no drug, efavirenz or UC781 and 0.1 OD600 units/ml of CTY10-5d expressing p66 bait and p51 prey fusions were grown in SC-His-Leu to OD₆₀₀=0.5 at 300 C. Cells were normalized to 12 OD₆₀₀ units and washed with 10 ml of TE (10 mM Tris pH 7.5; 1 mM EDTA) buffer. Preparation of protein extracts and immunoprecipitation were as previously described (30) except for the use of anti-HA.11 monoclonal antibodies (clone 16B12; Covance, Princeton, N.J.) and Protein G-PLUS agarose beads (Santa Cruz Biotechnology; Santa Cruz, Calif.). Samples were resolved by SDS-PAGE. The lexA87-66 fusion protein was probed using monoclonal antibodies 7E5 which specifically detects p66 (31).

In vitro Heterodimerization in the Presence of NNRTIs

The heterodimerization of bacterially expressed wild-type p66-His and GST-p51 (or mutants) was assessed in bacterial lysates as described previously (22). To determine the capacity of efavirenz to bind to a particular RT subunit, 500 μl reactions in lysis buffer (without NP-40) (22), 5 μg of p66-His, 5 μg GST-p51 or no recombinant protein (total protein concentration was 0.8 μg/ml in each reaction) were incubated overnight at 4° C. with increasing concentrations of efavirenz. Lysates were washed 4 times with lysis buffer using a centricon-YM-50 filter device (Millipore Corporation, Bedford, Mass.) to remove unbound drug. 5 μg of the corresponding RT subunit was applied to the washed lysates (in 500 μl) and incubated for 1.5 hr at 40C. Heterodimers were captured onto beads (22), resolved by SDS/PAGE and detected using RT antibodies (monoclonal antibody 5B2) (31).

Results

Enhancement of β-gal Activity by NNRTIs

To test the effects of NNRTIs on the association of the RT polypeptides we used a yeast genetic assay that measures RT heterodimerization (22). In this assay yeast expressing the p66 subunit of the HIV-1 RT fused to the C-terminus of lexa 87 (lexA87-66) and the p51 subunit fused to the Gal4AD (Gal4AD-51) constitutively interact, resulting in the activation of the expression from an integrated Lac Z reporter gene. To test for the effects of the NNRTIs on this interaction, 10 drugs representing 7 different NNRTI classes were added to the culture medium during growth of the yeast and β-gal levels were determined. Of the 10 NNRTIs tested, 9 demonstrated a dramatic concentration-dependent enhancement of β-gal activity compared to cells not treated with drug (FIGS. 7A and 7B). No significant toxicity, as determined by the growth rate, was observed for the drug concentrations tested compared to untreated controls (results not shown). Efavirenz was the most potent of the compounds, mediating a 40-fold increase in β-gal activity at the highest drug concentration tested (FIG. 7A). The carboxanilide UC781 was the second most potent drug, followed by UC10 and a quinoxaline, HBY 097 (FIGS. 7A and 7B). The remainder of the NNRTIs were less potent but still displayed 8-10 fold increases in 62 -gal activity at the highest concentrations tested (FIGS. 7A and 7B). In contrast delaviridine was devoid of P-gal enhancing activity (FIG. 7A).

Enhancement of β-gal Activity by NNRTIs is Specific for RT Heterodimerization

The specificity of the β-gal enhancement by NNRTIs was investigated. Yeast transformed with the empty vectors pSH21 and pGADNOT, which express lexA87 and Gal4AD, respectively were treated with serial dilutions of the most potent β-gal enhancing drug, efavirenz. We observed no increase in β-gal activity even in the presence of 15 μM of drug (data not shown). The capacity of efavirenz to enhance β-gal activity of several unrelated protein-protein interaction pairs, including moloney murine leukemia virus reverse transcriptase with elongation factor release factor 1 (M.O., unpublished), was also examined and no enhancement or inhibition of β-gal activity was observed.

The Y181C mutation in the p66 subunit of the HIV-1 RT confers more than a 100-fold increase in resistance to nevirapine (17). This mutation directly affects drug binding (13, 14). To further establish the specificity of the B-gal enhancement by NNRTIS, Y181C was introduced into the plasmid encoding the lexA87-66 fusion protein. Yeast were cotransformed with various pairs of plasmids and grown in the presence of nevirapine. The presence of the Y181C change in the p66 bait totally negated the enhancement effect by nevirapine (FIG. 8). In contrast, a significant level of β-gal enhancement was still retained in the presence of efavirenz (results not shown), consistent with the very low level of resistance conferred by Y181C to this drug. These data provide compelling evidence that the β-gal enhancement effect is due to a specific interaction of nevirapine with the p66 subunit of the HIV-1 RT.

NNRTIs can Enhance β-gal Activity of Dimerization Defective Mutants

Previous studies have shown that the L234A mutation in HIV-1 RT abrogates RT dimerization (22, 32). Other studies on the role of the tryptophan repeat motif (codons 398-414), present in the connection subdomains of both subunits, showed that the W401A mutation also diminishes RT dimerization in the Y2H assay (G.T. unpublished data). We investigated the effect of the NNRTIs, efavirenz and UC781 on the β-gal enhancement effect on these dimerization defective RT mutants. Interestingly, yeast treated with efavirenz and expressing the W401A change in one or both subunits showed a dramatic increase in β-gal activity compared to no drug (FIG. 9A). β-gal activity in yeast expressing the W401A mutation in both subunits was 100-fold greater in the presence of 1.6 μM efavirenz as compared with no drug (FIG. 9A). UC781 could also enhance the β-gal activity of yeast expressing W401A in both subunits (results not shown). It should be noted that while the mutants displayed large increases in β-gal activity, the actual activity (displayed on the top of each bar) did not significantly exceed that observed with wild-type RT grown in the presence of efavirenz.

The effect of efavirenz on the dimerization defective mutant L234A was also examined. Efavirenz enhanced β-gal activity in yeast expressing RT bait and prey fusions with L234A in one or both subunits (FIG. 9B). The fold increase in β-gal however, was less than with the W401A mutant (FIG. 9A). In contrast, UC781 conferred no enhancement on yeast expressing the L234A mutant (results not shown). As residue 234 is part of the NNRTI binding site (13), and may make contact with UC781 (33) it is possible that UC781 does not bind to the L234A mutant. These data indicate that NNRTIs are also able to suppress the defects in assembly caused by two mutations when they can bind to those mutant enzymes.

Coimmunoprecipitation of Heterodimer from Yeast Lysates Shows Enhancement of Dimer Formation in the Presence of Drug

Experiments suggest that neither an increase in steady-state protein levels of bait-prey fusions in yeast nor an increase in nuclear localization of the drug-heterodimer complex explain the observed enhancement of β-gal activity by NNRTIs (results not shown). To directly test for induced heterodimerization by NNRTIs we subjected lysates from yeast expressing dimerization defective mutants to coimmunoprecipitation. Yeast expressing both p66 bait and p51 prey fusions containing the W401A mutation (lexA₈₇-66W401A and Gal4AD-HA-51W401A) or the L234A change (lexA₈₇-66L234A and Gal4AD-HA-51L234A) were grown in the presence of efavirenz (1.6 μM), UC781 (16 μM) or no drug. Hemagglutinin (HA) antibodies were used to immunoprecipitate the p51 prey and the presence of any bound p66 bait was then detected using anti-p66 specific antibodies. For coimmunoprecipitation of the p66 bait fusions, samples were divided into two with one part processed without added NNRTI while the other was maintained in drug at the same concentration used during growth of yeast. Yeast grown in the absence of drug was also processed without drug or in the presence of 1.6 μM of efavirenz. A clear increase in the amount of lexA87-66W401A and lexA₈₇-66L234A associated with the p51 prey was observed for yeast grown in the presence of efavirenz compared to no drug (FIGS. 4A and 4B). Similar experiments with yeast grown in UC781 revealed heterodimer formation for yeast expressing the W401A mutant but not for the L234A mutant; these data corresponding to the levels of β-gal activity in the cells (FIGS. 4A and 4B).

No significant difference in the amount of coimmunoprecipitated p66 bait in the absence or presence of drug was observed indicating that the heterodimer was stable under the conditions of the assay. Interestingly, there was significantly more heterodimer present in yeast lysates obtained from cells grown in the absence of drug to which efavirenz was added during the coimmunoprecipitation procedure for the W401A mutant (FIG. 10A). These data suggest that some heterodimer formation could occur in vitro. Levels of mutant p66 bait and p51 prey fusions present in the original lysate from yeast grown in the absence and presence of drug were similar indicating that the increase in coimmunoprecipitated p66 bait in the presence of drug was not due to increased levels of fusion proteins. It is clear from these experiments that NNRTIs tested do act by inducing heterodimerization of p66 and p51 in the Y2H assay and that the increased dimer formation correlates with the increase in β-gal activity.

Efavirenz Enhances the Association of Wild-Type and Mutant p66 and p51 in Lysates in vitro

To explore whether NNRTIs could enhance dimerization in vitro, bacterial lysates containing either p66-His or GST-p51 were prepared and combined in the presence of increasing concentrations of efavirenz. In the absence of inhibitor a small amount of dimer was present as indicated by detectable amounts of p66-His. A concentration dependent increase in dimer formation was observed in the presence of increasing concentrations of efavirenz (FIG. 11). The enhancement effect of efavirenz on the L234A and W401A mutants was also assessed. Bacterial lysates separately expressing p66L234A-His and GST-p51L234A or p66W401A-His and GST-p51W401A were combined as above and incubated in the presence of increasing concentrations of efavirenz. A significant increase in dimer formation was observed in the presence of a 10-fold molar excess of efavirenz for the W401A mutant (FIG. 11). A 100-fold molar excess of efavirenz over RT was required to induce detectable enhancement of dimerization of the L234A mutant (FIG. 11). These data are consistent with the coimmunoprecipitation experiments and indicate that the enhancement of dimerization by efavirenz is due to its specific interaction with the HIV-1 RT and not dependent on the fusion proteins used in the Y2H assay nor on components present in the yeast cells in vivo.

Other NNRTIs Enhance Heterodimerization of RT Subunits in vitro

The in vitro study was extended by testing the remaining NNRTIs for their capacity to enhance the dimerization of GST-p51 and p66-His in vitro. Consistent with our Y2H data we observed that efavirenz was the most potent enhancer of dimerization. The relative in vitro potencies of the other NNRTIs correlated well with their β-gal enhancing effect in yeast (FIGS. 7 and 12). In contrast, UC781 and UC10 were poor dimerization inducers in bacterial lysates compared with their β-gal enhancing activities. The low dimerization enhancement activity of these drugs may be a function of both their poor solubility and the conditions of the in vitro assay (which was performed at 4° C.). In contrast, the conditions of the yeast assay, which was carried out at 30° C. with agitation, may have facilitated solubilization of UC781 and UC10. Interestingly, delavirdine was also inactive in vitro indicating that the lack of effect in yeast was not a result of the inability of this drug to penetrate the cells.

Efavirenz Enhances Heterodimerization by Binding to p66-His but not GST-p51

To help elucidate the mechanism by which efavirenz enhances heterodimerization we assessed whether this drug could bind to either p66-His or GST-p51. Bacterial lysates expressing p66-His, GST-p51 or no recombinant protein were preincubated in the absence or presence of increasing concentrations of efavirenz. Unbound drug was removed from the lysates by a series of washes and the presence of any remaining drug was assayed by the addition of the cognate RT subunit. p66-His and GST-p51 was added to a washed mock bacterial lysate to assess the efficiency of efavirenz removal. When p66-His was preincubated with efavirenz we observed enhancement of dimerization with subsequently added GST-p51 at all drug concentrations (FIG. 13). This enhancement was similar to controls where p66-His and GST-p51 were simultaneously combined with various drug concentrations (FIG. 12). A 100fold reduction in the potency of heterodimerization compared to p66-His preincubated with efavirenz was observed in the washed mock bacterial lysate (FIG. 13). GST-p51 preincubated with drug, washed and then subjected to the functional heterodimerization assay displayed the same pattern of heterodimerization observed for the drug-treated mock bacterial lysate. These data indicate that efavirenz binds tightly to p66-His but not GST-p51 and that this binding then promotes heterodimerization with subsequent added GST-p51.

Discussion

This study reports an important property of certain NNRTIs their capacity to enhance heterodimerization of the p66 and p51 subunits of the HIV-1 RT. This effect was observed both in the Y2H system, detecting dimerization of p66 and p51 using B-gal activity as a readout, and confirmed in coimmunoprecipitation experiments. The phenomenon was also observed in vitro using bacterially expressed GST-p51 and p66-His showing that it is not specific to yeast. NNRTIs were also able to induce the dimerization of the interaction defective mutants L234A and W401A. Furthermore, efavirenz can bind tightly to p66-His and then subsequently promote heterodimerization. The data indicate that NNRTIs have properties similar to conventional CIDs in their capacity to enhance the interaction between two proteins. As the interaction between p66 and p51 occurs naturally and the effect of the NNRTIs is to enhance this interaction then these small molecules are best described as chemical enhancers of dimerization.

Correlation Between in vitro and in vivo Enhancement of Heterodimerization by NNRTIs

The most potent β-gal enhancing NNRTIs in the Y2H RT dimerization assay were efavirenz, UC781 and HBY 097. These drugs are second generation NNRTIs that are also extremely potent inhibitors of HIV-1 replication in vitro (21, 25, 27). Efavirenz and UC781 differ from the other NNRTIs in that they bind very tightly to the RT heterodimer and exhibit very slow dissociation rates (Koff) (34, 35). The tight binding properties of efavirenz and UC781 may in part have contributed to their potency as enhancers of heterodimerization in yeast. There was generally a very good correlation between the relative potency in inducing dimerization of the NNRTIs in vitro and in yeast, with the exception of UC781 and UC10.

Relationship Between Drug Induced Enhancement of Dimerization, Structural Changes in the HIV-1 RT and RT Inhibitory Activity.

NNRTIs bind in a hydrophobic pocket at the base of the p66 thumb subdomain which is proximal to (˜10 Å), but distinct from the polymerase active. It is clear that the size of the NNRTI binding pocket is small compared to the extensive dimer interface (FIG. 14). No strong correlation was found between the extent of the p66/p51 interface (36, 37) in the structures of the HIV-1 RT in complex with several NNRTIs and the drug concentration mediating a 5-fold enhancement of β-gal activity. Thus, the NNRTI effect on heterodimerization is not a simple function of the surface area buried at the interface, and NNRTIs may affect dimerization by other mechanisms in addition to modulating the extent of the contacts. The position of the drug in the pocket and the degree of NNRTI interaction with the p51 subunit were found to vary significantly among the different RT/NNRTI complexes (FIG. 9), and the changes in the vicinity of the bound NNRTIs may also play a role in heterodimer formation.

Binding of efavirenz to RT is accompanied by conformational changes in the binding pocket region, and these changes (including at Leu234) (38), may also influence dimer formation. Delavirdine is the longest NNRTI inhibitor and a portion of it protrudes outside the NNRTI binding site causing the largest distortion of the p66 subunit of any of the NNRTIs studied to date (39). Delavirdine binds the furthest away from the p66/p51 interface (closest distance between delavirdine and p51 is 5.1 A compared to 3.8 Å for UC781) (FIG. 15). The unique characteristics of the interaction of delavirdine with HIV-1 RT suggest that this NNRTI may bind to p66 in a distinctive way that does not favor the enhancement of dimerization.

The relationship between the RT inhibitory activity of the NNRTIs in an exogenous RT assay (50% inhibitory concentration) and the concentration of drug that mediates a 5-fold increase in β-gal activity in the Y2H assay was compared. Efavirenz was the most potent in both assays while UC38 was the least active (results not shown). Examination of the data revealed a fair correlation (r=0.6) between these two parameters suggesting a relationship between the β-gal enhancement effect and RT inhibitory activity in vitro.

Potential Mechanisms for NNRTI Enhancement of Dimerization

How might the NNRTIs enhance heterodimerization? One possible model involves NNRTI binding directly to the p66 monomer. Drug binding to monomeric p66 may stabilize a conformation that is more conducive to heterodimer formation, and a more potent NNRTI may effectively increase the concentration of p66 in a conformation that promotes dimerization. Alternatively, efavirenz may cause the p66 monomer to have a conformational flexibility that allows this subunit to more readily undergo structural changes necessary for dimerization. A second model would entail NNRTIs binding only to the heterodimer and as a consequence stabilizing the dimer. The binding could shift the equilibrium toward the dimer. The data suggest that efavirenz binds tightly to p66. However, as bacterially expressed p66 comprises a population of monomers and homodimers it is unclear whether GST-p51 is binding directly to monomeric p66 complexed with drug or is exchanging with one p66 subunit in the drug bound homodimer. Elucidation of the exact mechanism of NNRTI induced enhancement of dimerization will require further studies.

The findings may have biological significance in terms of effects on virus replication. Drug binding to p66 could potentially modulate the interaction between Pr160^{GagPol} precursors which may affect regulation of HIV-1 protease-specific cleavage of this polyprotein. Further, the Y2H RT dimerization assay can potentially be used to screen for NNRTIs with the capacity to bind and mediate the appropriate conformational changes in the p66 subunit that results in enhanced binding to p51. It is possible that novel allosteric inhibitors of RT may be selected using this assay.

### REFERENCES FOR SECOND SERIES OF EXPERIMENTS

1. Goff, S. P. (1990) J. Acquir. Immune Defic. Syndr. 3, 817-831.

2. di Marzo Veronese, F., Copeland, T. D., DeVico, A. L., Rahman, R., Oroszlan, S., Gallo, R. C. & Sarngadharan, M. G. (1986) Science 231, 1289-1291.

3. Le Grice, S. F. J. (1993) in Reverse Transcriptase, eds. Skalka, A. M. & Goff, S. P. (Cold Spring Harbor Laboratory Press, Plainview), pp. 163-191.

4. Hsiou, Y., Ding, J., Das, K., Clark, A. D., Jr., Hughes, S. H. & Arnold, E. (1996) Structure 4, 853-860.

5. Kohlstaedt, L. A., Wang, J., Friedman, J. M., Rice, P. A. & Steitz, T. A. (1992) Science 256, 1783-1790.

6. Das, K., Ding, J., Hsiou, Y., Clark, A. D., Jr., Moereels, H., Koymans, L., Andries, K., Pauwels, R., Janssen, P. A., Boyer, P. L. & et al. (1996) J. Mol. Biol. 264, 1085-1100.

7. Huang, H., Chopra, R., Verdine, G. L. & Harrison, S.C. (1998) Science 282, 1669-1675.

8. Jacobo-Molina, A., Ding, J., Nanni, R. G., Clark, A. D., Jr., Lu, X., Tantillo, C., Williams, R. L., Kamer, G., Ferris, A. L., Clark, P. & et al. (1993) Proc. Natl. Acad. Sci. U S A 90, 6320-6324.

9. Becerra, S. P., Kumar, A., Lewis, M. S., Widen, S. G., Abbotts, J., Karawya, E. M., Hughes, S. H., Shiloach, J. & Wilson, S. H. (1991) Biochemistry 30, 11707-11719.

10. Wang, J., Smerdon, S. J., Jager, J., Kohlstaedt, L. A., Rice, P. A., Friedman, J. M. & Steitz, T. A. (1994) Proc. Natl. Acad. Sci. USA 91, 7242-7246.

11. De Clercq, E. (1998) Antiviral Res. 38, 153-179.

12. Pedersen, 0. S. & Pedersen, E. B. (1999) Antivir. Chem. Chemother. 10, 285-314.

13. Smerdon, S. J., Jager, J., Wang, J., Kohlstaedt, L. A., Chirino, A. J., Friedman, J. M., Rice, P. A. & Steitz, T. A. (1994) Proc. Natl. Acad. Sci. U S A 91, 3911-3915.

14. Tantillo, C., Ding, J., Jacobo-Molina, A., Nanni, R. G., Boyer, P. L., Hughes, S. H., Pauwels, R., Andries, K., Janssen, P. A. & Arnold, E. (1994) J. Mol. Biol. 243, 369-387.

15. Ding, J., Das, K., Tantillo, C., Zhang, W., Clark, A. D., Jr., Jessen, S., Lu, X., Hsiou, Y., Jacobo-Molina, A., Andries, K. & et al. (1995) Structure 3, 365-379.

16. Esnouf, R., Ren, J., Ross, C., Jones, Y., Stammers, D. & Stuart, D. (1995) Nat. Struct. Biol. 2, 303-308.

17. Richman, D., Shih, C. K., Lowy, I., Rose, J., Prodanovich, P., Goff, S. & Griffin, J. (1991) Proc. Natl. Acad. Sci. U S A 88, 11241-11245.

18. Richman, D. D., Havlir, D., Corbeil, J., Looney, D., Ignacio, C., Spector, S. A., Sullivan, J., Cheeseman, S., Barringer, K., Pauletti, D. & et al. (1994) J. Virol. 68, 1660-1666.

19. Merluzzi, V. J., Hargrave, K. D., Labadia, M., Grozinger, K., Skoog, M., Wu, J. C., Shih, C. K., Eckner, K., Hattox, S., Adams, J. & et al. (1990) Science 250, 14111413.

20. Romero, D. L., Busso, M., Tan, C. K., Reusser, F., Palmer, J. R., Poppe, S. M., Aristoff, P. A., Downey, K. M., So, A. G., Resnick, L. & et al. (1991) Proc. Natl. Acad. Sci. U S A 88, 8806-8810.

21. Young, S. D., Britcher, S. F., Tran, L. O., Payne, L. S., Lumma, W. C., Lyle, T. A., Huff, J. R., Anderson, P. S., Olsen, D. B., Carroll, S. S. & et al. (1995) Antimicrob. Agents Chemother. 39, 2602-2605.

22. Tachedjian, G., Aronson, H. E. & Goff, S. P. (2000) Proc. Natl. Acad. Sci. U S A 97, 6334-6339.

23. Crabtree, G. R. & Schreiber, S. L. (1996) Trends Biochem. Sci. 21, 418-422.

24. Balzarini, J., Brouwer, W. G., Felauer, E. E., De Clercq, E. & Karlsson, A. (1995) Antiviral Res. 27, 219-236.

25. Balzarini, J., Brouwer, W. G., Dao, D. C., Osika, E. M. & De Clercq, E. (1996) Antimicrob. Agents Chemother. 40, 1454-1466.

26. Dueweke, T. J., Poppe, S. M., Romero, D. L., Swaney, S. M., So, A. G., Downey, K. M., Althaus, I. W., Reusser, F., Busso, M., Resnick, L. & et al. (1993) Antimicrob. Agents Chemother. 37, 1127-1131.

27. Kleim, J. P., Bender, R., Kirsch, R., Meichsner, C., Paessens, A., Rosner, M., Rubsamen-Waigmann, H., Kaiser, R., Wichers, M., Schneweis, K. E. & et al. (1995) Antimicrob. Agents Chemother. 39, 2253-2257.

28. Ho, W., Kukla, M. J., Breslin, H. J., Ludovici, D. W., Grous, P. P., Diamond, C. J., Miranda, M., Rodgers, J. D., Ho, C. Y., De Clercq, E. & et al. (1995) J. Med. Chem. 38, 794-802.

29. Pauwels, R., Andries, K., Debyser, Z., Van Daele, P., Schols, D., Stoffels, P., De Vreese, K., Woestenborghs, R., Vandamme, A. M., Janssen, C. G. & et al. (1993) Proc. Natl. Acad. Sci. U S A 90, 1711-1715.

30. Kuchin, S., Treich, I. & Carlson, M. (2000) Proc. Natl. Acad. Sci. U S A 97, 7916-7920.

31. Szilvay, A. M., Nornes, S., Haugan, I. R., Olsen, L., Prasad, V. R., Endresen, C., Goff, S. P. & Helland, D. E. (1992) J. Acquir. Immune Defic. Syndr. 5, 647-657.

32. Ghosh, M., Jacques, P. S., Rodgers, D. W., Ottman, M., Darlix, J. L. & Le Grice, S. F. (1996) Biochemistry 35, 8553-8562.

33. Esnouf, R. M., Stuart, D. I., De Clercq, E., Schwartz, E. & Balzarini, J. (1997) Biochem. Biophys. Res. Commun. 234, 458-464.

34. Barnard, J., Borkow, G. & Parniak, M. A. (1997) Biochemistry 36, 7786-7792.

35. Maga, G., Ubiali, D., Salvetti, R., Pregnolato, M. & Spadari, S. (2000) Antimicrob. Agents Chemother. 44, 1186-1194.

36. Arnold, E. & Rossmann, M. G. (1990) J. Mol. Biol. 211, 763-801.

37. Lee, B. & Richards, F. M. (1971) J. Mol. Biol. 55, 379-400.

38. Ren, J., Milton, J., Weaver, K. L., Short, S. A., Stuart, D. I. & Stammers, D. K. (2000) Structure Fold Des. 8, 1089-1094.

39. Esnouf, R. M., Ren, J., Hopkins, A. L., Ross, C. K., Jones, E. Y., Stammers, D. K. & Stuart, D. I. (1997) Proc. Natl. Acad. Sci. U S A 94, 3984-3989.

What is claimed:

1. A method of determining whether a compound enhances formation of a complex between a wild-type p66 subunit polypeptide of HIV-1 reverse transcriptase and a wild-type p51 subunit polypeptide of HIV-1 reverse transcriptase and/or enhances formation of a complex between a p66 subunit having at least one mutation associated with resistance of HIV-1 to at least one nonnucleoside reverse transcriptase inhibitor (NNRTI) and a wild-type p51 subunit of HIV-1 reverse transcriptase, said method comprises:
a) contacting a yeast cell with the compound, which cell comprises (i) a first plasmid which expresses a fusion protein comprising a wild-type p66 subunit polypeptide of HIV-1 reverse transcriptase, (ii) a second plasmid which expresses a fusion protein comprising a wild-type p51 subunit polypeptide of HIV-1 reverse transcriptase, and (iii) a reporter gene which is activated in the presence of a complex between the wild-type p66 subunit polypeptide and the wild-type p51 subunit polypeptide, and determining the level of activity of the reporter gene in the cell in the presence of the compound; and
b) comparing the level of activity of the reporter gene determined in step (a) with a level of activity of the reporter gene determined in the absence of the compound, wherein an increased level of activity of the reporter gene determined in step (a) indicates that the compound is an activator of the formation of the complex between the wild-type p51 subunit polypeptide of HIV-1 reverse transcriptase and the wild-type p66 subunit polypeptide of HIV-1 reverse transcriptase; and
c) contacting a yeast cell with the compound, which cell comprises (i) a third plasmid which expresses a fusion protein comprising a p66 subunit polypeptide of HIV-1 reverse transcriptase having at least one mutation associated with resistance of HIV-1 to at least one NNRTI, (ii) a second plasmid which expresses a fusion protein comprising a wild-type p51 subunit polypeptide of HIV-1 reverse transcriptase, and (iii) a reporter gene which is activated in the presence of a complex between the p66 subunit polypeptide having at least one mutation and the wild-type p51 subunit polypeptide, and determining the level of activity of the reporter gene in the cell in the presence of the compound; and
d) comparing the level of activity of the reporter gene determined in step (c) with a level of activity of the reporter gene determined in the absence of the compound, wherein an increased level of activity of the reporter gene determined in step (c) indicates that the compound is an activator of the formation of the complex between the wild-type p51 subunit polypeptide of HIV-1 reverse transcriptase and the p66 subunit polypeptide of HIV-1 reverse transcriptase having at least one mutation associated with resistance of HIV-1 to at least one NNRTI.

2. The method according to claim 1, wherein the mutation is associated with at least 5-fold or greater resistance of HIV-1 to at least one NNRTI.

3. The method according to claim 1, wherein the mutation is associated with at least a ten-fold or greater resistance of HIV-1 to at least one NNRTI.

4. The method according to claim 1, wherein the p66 subunit polypeptide has one mutation, the mutation is associated with an increase in resistance of HIV-1 to at least one NNRTI.

5. The method according to claim 1, wherein the p66 subunit polypeptide has two mutations, each mutation is associated with an increase in resistance of HIV-1 to at least one NNRTI.

6. The method according to claim 1, wherein the p66 subunit polypeptide has three mutations, each mutation is associated with an increase in resistance of HIV-1 to at least one NNRTI.

7. The method according to claim 1, wherein the p66 subunit polypeptide has four mutations, each mutation is associated with an increase in resistance of HIV-1 to at least one NNRTI.

8. The method according to claim 1, wherein the p66 subunit polypeptide has five mutations, each mutation is associated with an increase in resistance of HIV-1 to at least one NNRTI.

9. The method according to claim 1, wherein the p66 subunit polypeptide mutation is F227L, G190A, G190E, G190S, K101E, K103N, K238T, L100I, P225H, V106A, V106I, V108I, Y181C, Y188H, Y188L, or combinations thereof.

10. The method according to claim 9, wherein the p66 subunit polypeptide mutation is L1001 and K103N.

11. The method according to claim 9, wherein the p66 subunit polypeptide mutation is selected from the group consisting of, K103N and Y181C; K101E and K103N; K103N and Y188H; V106I and Y188L; K103N and P225H; and K103N and V108I.

12. The method according claim 9, wherein a first mutation is K103N or Y188L and a second mutation is selected from the group consisting of V106I, V108I, Y181C, Y188H, P225H and F227L.

13. The method according to claim 1, wherein the yeast strain is CTY10-5d having the genotype: MATa ade2 trp1-901 leu2-3112 his3-200 gal4-gal80- URA3::lexA-lacZ.

14. A compound determined to be capable of enhancing formation of a complex between a p66 subunit polypeptide of HIV-1 reverse transcriptase having at least one mutation and a wild-type p51 subunit polypeptide of HIV-1 reverse transcriptase by the method of claim 1.

15. The compound according to claim 14, wherein the compound inhibits HIV-1.

16. The compound according to claim 14, wherein the enhanced formation of complex formation by the compound is measured by enhancement of the activity of the reporter gene.

17. The compound according to claim 14, wherein the p66 subunit polypeptide of HIV-1 reverse transcriptase has one mutation, the mutation is associated with an increased resistance of HIV-1 to at least one NNRTI.

18. The compound according to claim 14, wherein the p66 subunit polypeptide of HIV-1 reverse transcriptase has two mutations, each mutation is associated with an increase in resistance of HIV-1 to at least one NNRTI.

19. The compound according to claim 14, wherein the p66 subunit polypeptide of HIV-1 reverse transcriptase has three mutations, each mutation is associated with an increase in resistance of HIV-1 to at least one NNRTI.

20. The compound according to claim 14, wherein the p66 subunit polypeptide of HIV-1 reverse transcriptase has four mutations, each mutation is associated with an increase in resistance of HIV-1 to at least one NNRTI.

21. The compound according to claim 14, wherein the p66 subunit polypeptide of HIV-1 reverse transcriptase has five mutations, each mutation is associated with an increase in resistance of HIV-1 to at least one NNRTI.

22. The compound according to claim 14, wherein the mutation is F227L, G190A, G190E, G190S, K101E, K103N, K238T, L1001, P225H, V106A, V106I, V108I, Y181C, Y188H, Y188L, or combinations thereof.

23. The compound according to claim 22, wherein the mutation is selected from the group consisting of, L1001 and K103N; Y181C and K103N; K101E and K103N; K103N and Y188H; V106I and Y188L; 103N and P225H, and K103N and V108I.

24. The compound according to claim 22, wherein a first mutation is K103N or Y188L and a second mutation is selected from the group consisting of V106I, V108I, Y188H, P225H, and F227L.

25. The compound according to claim 14, wherein an enhancement of formation of a complex between a p66 subunit polypeptide of HIV-1 reverse transcriptase having at least one mutation associated with an increased resistance of HIV-1 to at least one NNRTI and a wild-type p51 subunit polypeptide of HIV-1 reverse transcriptase is comparable to an enhancement of formation of a complex between a wild-type p66 subunit polypeptide of HIV-1 reverse transcriptase and a wild-type p51 subunit polypeptide of reverse transcriptase at a given compound concentration.

26. The compound according to claim 25, wherein the enhancement obtained using the p66 subunit having at least one mutation associated with an increase in resistance of HIV-1 to at least one NNRTI and the enhancement obtained using wild-type p66 are within plus-or-minus 15%.

27. The compound according to claim 25, wherein the formation of complex by the compound using mutant p66 or wild-type p66 is greater than formation of complex by a known NNRTI using mutant p66 or wild-type at a given concentration, that concentration being in a linear range of enhancement effects for both the compound and the known NNRTI.

28. The compound according to claim 14, which has little or no enhancement of formation of complex between the wild-type p66 subunit polypeptide and the wild-type p51 subunit polypeptide.

29. The compound according to claim 14, wherein the enhancement of formation of complex between the p66 subunit having at least one mutation associated with an increase in resistance of HIV-1 to at least one NNRTI and wild-type p51 subunit is greater than the enhancement of formation of complex between wild-type p66 subunit and wild-type p51 subunit.

30. The compound according to claim 29, wherein the enhancement of complex formation between the p66 subunit having at least one mutation associated with an increase in resistance of HIV-1 to at least one NNRTI and wild-type p51 subunit is greater than 25% of the enhancement of complex formation between wild-type p66 subunit and wild-type p51 subunit.

31. The compound according to claim 29, wherein the enhancement of complex formation between the p66 subunit having at least one mutation associated with an increase in resistance of HIV-1 to at least one NNRTI and wild-type p55 subunit is at least 30% of the enhancement of complex formation between wild-type p66 subunit and wild-type p51 subunit.

32. The compound according to claim 29, wherein the enhancement of complex formation between the p66 subunit having at least one mutation associated with an increase in resistance of HIV-1 to at least one NNRTI and wild-type p51 subunit is at least 50% of the enhancement of complex formation between the wild-type p66 subunit and wild-type p51 subunit.

33. The compound according to claim 29, wherein the enhancement of complex formation between the p66 subunit having at least one mutation associated with an increase in resistance of HIV-1 to at least one NNRTI and wild-type p51 subunit is at least 75% of the enhancement of complex formation between the wild-type p66 subunit and wild-type p51 subunit.

34. The compound according to claim 29, wherein the enhancement of complex formation between p66 subunit having at least one mutation associated with an increase in resistance of HIV-1 to at least one NNRTI and wild-type p51 subunit is about 30% to about 100% of the enhancement of complex formation between wild-type p66 subunit and wild-type p51 subunit.

35. The compound according to claim 29, wherein the enhancement of complex formation between p66 subunit having at least one mutation associated with an increase in resistance of HIV-1 to at least one NNRTI and wild-type p51 subunit is about 40% to about 100% of the enhancement of complex formation between wild-type p66 subunit and wild-type p51 subunit.

36. The compound according to claim 29, wherein the enhancement of complex formation between p66 subunit having at least one mutation associated with an increase in resistance of HIV-1 to at least one NNRTI and wild-type p51 subunit is about 50% to about 100% of the enhancement of complex formation between wild-type p66 subunit and wild-type p51 subunit.

37. The compound according to claim 29, wherein the enhancement of complex formation between p66 subunit having at least one mutation associated with an increase in resistance of HIV-1 to at least one NNRTI and wild-type p51 subunit is about 100% to about 500% of the enhancement of complex formation between wild-type p66 subunit and wild-type p51 subunit.

38. The compound according to claim 29, wherein the enhancement of complex formation between p66 subunit having at least one mutation associated with an increase in resistance of HIV-1 to at least one NNRTI and wild-type p51 subunit is about 150% to about 1000% of the enhancement of complex formation between wild-type p66 subunit and wild-type p51 subunit.

39. The compound according to claim 29, wherein the enhancement of complex formation between p66 subunit having at least one mutation associated with an increase in resistance of HIV-1 to at least one NNRTI and wild-type p51 subunit is about 200% to about 1000% of the enhancement of complex formation between wild-type p66 subunit and wild-type p51 subunit.

40. The compound according to claim 29, wherein the enhancement of complex formation between p66 subunit having at least one mutation associated with an increase in resistance of HIV-1 to at least one NNRTI and wild-type p51 subunit is about 500% to about 1000% of the enhancement of complex formation between wild-type p66 subunit and wild-type p51 subunit.

41. The compound according to claim 29, wherein the p66 subunit has at least one mutation and the enhancement of complex formation between the p66 subunit having the mutation and the wild-type p51 subunit is higher than the enhancement of complex formation in the presence of a known NNRTI at a given concentration, that concentration being in a linear range of enhancement for both the compound and the known NNRTI.

42. The compound according to claim 29, wherein the p66 subunit has at least one mutation and the enhancement of complex formation between the p66 subunit having the mutation and the wild-type p51 subunit is higher than the enhancement of complex formation in the presence of (S)6-chloro-4-(cyclopropylethynyl)-1,4-dihydro-4-(trifluoromethyl)-2H-3,1-benzoxazin-2-one (efavirenz) at a given concentration, that concentration being in a linear range of enhancement for both the compound and efavirenz.

43. The compound according to claim 42, wherein the enhancement of complex formation is at least 20% higher than the enhancement in complex formation in the presence of efavirenz at a given concentration, that concentration being in a linear range of enhancement for both the compound and efavirenz.

44. The compound according to claim 42, wherein the enhancement of complex formation is at least 25% higher than the enhancement of complex formation in the presence of efavirenz at a given concentration, that concentration being in a linear range of enhancement for both the compound and efavirenz.

45. The compound according to claim 42, wherein the enhancement of complex formation is at least 30% higher than the enhancement of complex formation in the presence of efavirenz at a given concentration, that concentration being in a linear range of enhancement for both the compound and efavirenz.

46. The compound according to claim 42, wherein the enhancement of complex formation is at least 50% higher than the enhancement of complex formation in the presence of efavirenz at a given concentration, that concentration being in a linear range of enhancement for both the compound and efavirenz.

47. The compound according to claim 42, wherein the enhancement of complex formation is about 50% to about 1000% higher than the enhancement of complex formation in the presence of efavirenz at a given concentration, that concentration being in a linear range of enhancement for both the compound and efavirenz.

48. The compound according to claim 42, wherein the enhancement of complex formation is about 100% to about 1000% higher than the enhancement of complex formation in the presence of efavirenz at a given concentration, that concentration being in a linear range of enhancement for both the compound and efavirenz.

49. The compound according to claim 42, wherein the enhancement of complex formation is about 150% to about 1000% higher than the enhancement of complex formation in the presence of efavirenz at a given concentration, that concentration being in a linear range of enhancement for both the compound and efavirenz.

50. The compound according to claim 42, wherein the enhancement of complex formation is about 200% to about 1000% higher than the enhancement of complex formation in the presence of efavirenz at a given concentration, that concentration being in a linear range of enhancement for both the compound and efavirenz.

51. The compound according to claim 42, wherein the enhancement of complex formation is about 500% to about 1000% higher than the enhancement of complex formation in the presence of efavirenz at a given concentration, that concentration being in a linear range of enhancement for both the compound and efavirenz.

52. The compound of claim 14, wherein the compound is a nonnucleoside reverse transcriptase inhibitor.

53. The compound according to claim 14, wherein the compound is capable of inhibiting the growth of HIV-1.

54. The compound according to claim 53, wherein the HIV-1 is resistant to at least one NNRTI.

55. The compound according to claim 53, wherein the HIV-1 has at least one mutation in reverse transcriptase, said mutation is associated with resistance to at least one NNRTI.

56. The compound according to claim 54 or 55, wherein the NNRTI is known.

57. The compound of claim 1 4, wherein the compound is not efavirenz, N- (4-chloro-3-(3-methyl-2-butenyloxy)phenyl)-2-methyl-3-furancarbothioamide (UC-781), nevirapine, delavirdine, SJ-3366, MKC-442, GW420867x or (S)-4-isopropoxycarbonyl-6-methoxy-3-(methylthiomethyl)-3,4-dihydroquinoxaline-2(1H)-thione (HBY 097).

58. The compound according to claim 14, wherein the compound is a derivative of a compound selected from the group consisting of efavirenz, UC-781, HBY 097, nevirapine, delavirdine, SJ-3366, MKC-442, GW420867x, and HI-443.

59. The compound according to claim 14, wherein the compound acts at a location on HIV-1 reverse transcriptase distinct from a nonnucleoside reverse transcriptase binding pocket.

60. A compound capable of inhibiting HIV-1, said inhibition characterized by enhancing formation of a complex between a p66 subunit polypeptide and a p51 subunit polypeptide of reverse transcriptase of the HIV-1, wherein the HIV-1 is wild-type or the HIV-1 has at least one mutation associated with resistance to at least one NNRTI.

61. The compound according to claim 60, wherein the mutation is in the p66 subunit and the p51 subunit of reverse transcriptase.

62. The compound according to claim 60, wherein the mutation is F227L, G190A, G190E, G190S, K101E, K103N, K238T, L100I, P225H, V106A, V106T, V108I, Y181C, Y188H, Y188L, or combinations thereof.

63. The compound according to claim 60, wherein the mutation is selected from the group consisting of K103N, and Y181C; K101E and K103N; K 103N and Y188H; V106I and Y188L; K103N and P225H; and K103N and V108I.

64. The compound according to claim 60, wherein a first mutation is K103N or Y188L and a second mutation is selected from the group consisting of V106I, V108I, Y181C, Y188H, P225H and F227L.

65. The compound according to claim 60, wherein the compound is derived from a combinatorial chemical library.

66. The compound according to claim 60, wherein the compound is derived from a synthetic chemical library.

67. The compound according to claim 60, wherein the compound is a nonpeptidyl agent having a molecular weight of less than 1000 daltons.

68. The compound according to claim 60, wherein the compound is an NNRTI.

69. The compound according to claim 68, wherein the compound is a derivative of efavirenz, UC-781, HBY 097, nevirapine, delavirdine, SJ-3366, MKC-442, GW420867x or HI-443.

70. The compound according to claim 60, wherein the compound has an IC₅₀ value against growth of HIV-1 of less than one nanomolar.

71. The compound according to claim 60, wherein the compound has an IC₅₀ value against the growth of HIV-1 in a range of about 0.01 nM to about 1.0 nM.

72. A composition comprising at least one compound according to any one of claims 16 or 60 and a pharmaceutically acceptable carrier.

73. The composition according to claim 72, further comprising at least one known NNRTI, a nucleoside reverse transcriptase inhibitor, an HIV-1 protease inhibitor, or combinations thereof.

74. The composition according to claim 73, wherein the known NNRTI is selected from the group consisting of efavirenz, UC-781, HBY 097, nevirapine, delavirdine, SJ-3366, MKC-442, GW420867x, and HI-443 and combinations thereof.

75. The composition according to claim 73, wherein the nucleoside reverse transcriptase inhibitor is selected from the group consisting of abacavir, lamivudine, zidovudine, stavudine, zalcitabine, didanosine, and combinations thereof.

76. The compositions according to claim 73, wherein the HIV-1 protease inhibitor is selected from the group consisting of lopinavir, saquinavir, nelfinavir, indinavir, amprenavir, ritonavir, and combinations thereof.

77. A method of enhancing formation of a complex between a p66 subunit polypeptide of HIV-1 reverse transcriptase and a p51 subunit polypeptide of reverse transcriptase, which comprises contacting the subunits with an effective amount of at least one compound according to claim 14, so to thereby enhance formation of a complex between the p66 subunit polypeptide and the p51 subunit polypeptide of HIV-1 reverse transcriptase.

78. The method according to claim 77, wherein the HIV-1 reverse transcriptase has at least one mutation associated with resistance to at least one NNRTI.

79. The method according to claim 77, wherein the HIV-1 reverse transcriptase is present in a subject and the contacting is effected by administering the compound to the subject.

80. The method of claim 79, wherein the compound is administered orally, intravenously, subcutaneously, intramuscularly, topically, of by liposome-mediated delivery.

81. The method according to claim 79, wherein the subject is a human, a non-human primate, an equine, an opine, an avian, a bovine, a porcine, a canine, a feline, or a mouse.

82. The method according to claim 79, wherein the effective amount of the compound is between about 1 mg and about 50 mg per kg body weight of the subject.

83. The method according to claim 79, wherein the effective amount of the compound is between about 2 mg and about 40 mg per kg body weight of the subject.

84. The method according to claim 79, wherein the effective amount of the compound is between about 3 mg and about 30 mg per kg body weight of the subject.

85. The method according to claim 79, wherein the effective amount of the compound is about 4 mg and about 20 mg per kg body weight of the subject.

86. The method according to claim 79, wherein the effective amount of the compound is between about 5 mg and about 10 mg per kg body weight of the subject.

87. The method according to claim 79, wherein the compound is administered at least once per day.

88. The method according to claim 79, wherein the compound is administered daily.

89. The method according to claim 79, wherein the compound is administered every other day.

90. The method according to claim 79, wherein the compound is administered every 6 to 8 days.

91. The method according to claim 79, wherein the compound is administered weekly.

92. A method of inhibiting the growth of HIV-1 comprising administration of at least one compound according to claim 14 or 60, in an amount effective to inhibit HIV-1.

93. The method according to claim 92, wherein the HIV-1 has at least one mutation in subunit polypeptides of reverse transcriptase.

94. The method according to claim 93, wherein a p51 subunit polypeptide of reverse transcriptase of the HIV-1 has a mutation corresponding to a mutation in a p66 subunit polypeptide.

95. The method according to claim 92, wherein the HIV-1 is resistant to at least one NNRTI.

96. The method according to claim 92, further comprising the administration of at least one known NNRTI, a nucleoside reverse transcriptase inhibitor, an HIV-1 protease inhibitor, or combinations thereof.

97. A method of treating infection by HIV-1 or for treating AIDS in a subject comprising administration of an amount of at least one compound according to claim 14 or 60, said amount effective to treat the infection or AIDS.

98. The method according to claim 97, wherein the HIV-1 in the subject has at least one mutation in a reverse transcriptase gene.

99. The method according to claim 97, wherein the HIV-1 viral load of the subject has increased with conventional NNRTI therapy.

100. A method of making a pharmaceutical composition which comprises:
a) determining whether a compound enhances formation of complex by the method of claim 1;
b) recovering the compound; and
c) admixing the compound with a pharmaceutically acceptable carrier.
